(19) United States
(12) Patent Application Publication
(10) Pub. No.: US 2010/0184703 A1
(43) Pub. Date: Jul. 22, 2010

(54) USE OF PEPTIDE DERIVATIVES FOR TREATING PATHOLOGIES RESULTING FROM ISCHEMIA

(75) Inventors: Richard Casimir, Vaires sur Marne (FR); David Chauvier, Limeil-Brevannes (FR); Etienne Jacotot, Paris (FR); Alain Lacampagne, Vendargues (FR); Jeremy Fauconnier, Montpellier (FR)

Correspondence Address: OBLON, SPIVAK, MCCLELLAND MAIER & NEUSTADT, L.L.P., 1940 DUKE STREET, ALEXANDRIA, VA, 22314, US

(73) Assignees: CHIESI FARMACEUTICI S.p.A., PARMA (IT); INST. NAT. DE LA SANTE ET DE LA RECH. MED.(INSERM), PARIS (FR)

(21) Appl. No.: 12/666,462

(22) PCT Filed: Jun. 27, 2008
(86) PCT No.: PCT/IB08/52591

§ 371 Date: Dec. 23, 2009

Related U.S. Application Data
(60) Provisional application No. 60/937,406, filed on Jun. 27, 2007.

(51) Int. Cl. A61K 38/08; C07K 7/06; A61P 9/10

(52) U.S. Cl. 514/17; 530/330

(57) ABSTRACT

The inventors have studied the kinetic and hierarchy of activation of apoptogenic caspases during myocardial ischemia and have found that caspase 2 plays a major role during the cardiac pathology by a very fast activation after an ischemic episode. Experiments were carried out on animal models of transitory or permanent myocardial ischemia. It was then observed that electrophysiological remodeling and post-ischemic fibrosis were prevented by using caspase-2 specific inhibitors. The invention is then based on the demonstration that caspase-2 and its activation represents an early and transitory step of the cardiac apoptotic mechanisms resulting from a myocardial ischemic and involved in the development of hypertrophy and cardiac insufficiency. The invention thus relates to a method of treatment of cardiovascular pathologies resulting from ischemia by using certain caspase-2 specific inhibitors.

The invention relates to the use of peptide derivatives for treating pathologies resulting from ischemia.

It also relates to new peptide derivatives and their biological applications.

The invention particularly relates to the treatment of cardiovascular pathologies resulting from ischemia.

Cardiovascular diseases are today in progress in a large number of developing countries where they become the main cause of mortality.

In industrialized countries, the prevention steps enable a slowing down of the progress of these diseases which however remain the first cause of mortality.

It has to be noted that more than a third of the deaths concern old individuals. As the general ageing of the population is progressing, there is a real need in identifying the cause(s) of cardiac insufficiency, more especially their earliest causes. In that respect, the identification of the factors involved in the process resulting in heart failure or cardiac insufficiency is essential.

In Western countries, myocardial ischemic represents in more than a third of the cases the cause of myocardial infarctus. In South America countries, frequent causes are myocardites such as Chagas disease or viral myocardites. Inflammatory myocardites of acute rheumatoid arthritis are also frequent in developing countries.

Myocardia infarctus corresponds to a decrease of oxygen supply to the cells of the cardiac muscle which results in their death and destruction of a part of the cardiac muscle. The loss of cardiomyocytes has for a long time been mainly attributed to cellular necrosis processus. It was further demonstrated that cardiomyocytes could also die by apoptosis. This observation was confirmed in different cardiopathies (ischemic, hypertrophic, dilated and other cardiopathies). Today, apoptosis is considered as an important physiopathological mechanism in cardiology.

It was then postulated that apoptosis of cardiomyocytes could be a causal mechanism of evolution toward cardiac insufficiency and that the inhibition of these death mechanisms were a major stake for developing novel cardio therapies.

Apoptosis occurs through a cascade of cellular and sub-cellular events, such as cytochrome C release mitochondrial to cytoplasm and activation of a series of cystein proteases, i.e. caspases.

By now about 15 caspases have been identified in mammals.

Briefly, the activation of caspases results in the fragmentation of cytoplasmic proteins, including the contractile machinery. It was then postulated that the cytochrome C release and the proteic degradation widely contributes to the cystolic degradation.

But damages at the nuclear level which usually characterized the final stage of apoptosis are rather rare in cardiac insufficiency situations. In that respect, in heart, it is referred to “apoptosis interruptus” which corresponds to partial protection of nuclear material by apoptotic mechanism, enabling a certain cytoplasm reconstitution.

It is then proposed that proapoptotic mechanisms contribute to the cellular death but also to the structural and functional remodelling which inexorably contribute to the progression of the pathology.

The interruption or inhibition of proapoptic mechanisms then appears to be a therapeutical route of great interest to limit and even prevent cardiac insufficiency evolution.

Attempts to block proapoptotic mechanisms or animal models have been reported. On rat, for example, non specific inhibition of caspases or specific caspases 8, 9 and 3 inhibitions significantly resulted in an inhibition of the infarcted zone extent induced by an ischemia of 35 min. (Mocanu, 2000). However, the efficiency of such a treatment over a long period of time has not yet been confirmed.

The identification of the apoptosis route in the myocardium during ischemia is still incomplete nowadays.

An extracellular route essentially represented by the increase of inflammation markers (cytokine) is reported to activate an intermediary caspases.

By now, caspase 8 appears to be the only one which has been identified. In the same way, it has been reported that the same extracellular routes would activate cytochrome C release which in turn would activate caspase 9. Both routes would result in caspase 3 activation. Other caspases have been described in heart, but their role has not been defined.

The inventors have studied the kinetic and hierarchy of activation of apoptogenic caspases during myocardial ischemia and have found that caspase 2 plays a major role during the cardiac pathology by a very fast activation after an ischemic episode.

Experiments were carried out on animal models of transitory or permanent myocardial ischemia. It was then observed that electrophysiological remodeling and post-ischemic fibrosis were prevented by using caspase-2 specific inhibitors.

The invention is then based on the demonstration that caspase-2 and its activation represents an early and transitory step of the cardiac apoptotic mechanisms resulting from a myocardial ischemic and involved in the development of hypertrophy and cardiac insufficiency.

The invention thus relates to a method of treatment of cardiovascular pathologies resulting from ischemia.

It also relates to the use of caspase-2 inhibitors for making drugs for such a treatment.

Another object of the invention is to provide new caspase-2 inhibitors of great therapeutical value.

Still another object is to provide drugs comprising the new caspase-2 inhibitors as active principles.

The invention thus relates to the use of caspase 2-specific inhibitor for treating cardiovascular pathologies resulting from ischemic situations.

More specifically, the invention relates to the use wherein the caspase 2-specific inhibitor is a derivative of formula I

R—CO-A1-AspSubst-A-AspSubst-R1-R2 (I)

wherein
R is selected in the group comprising
a quinolin-2-yl group of formula II

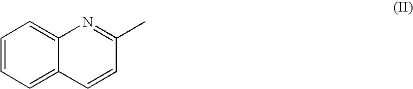

or,
substituted phenyl group of formula III

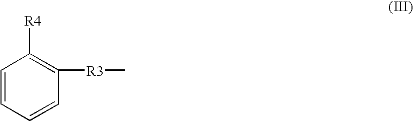

with R3 being —NH—CO— or —NH—CO—CH₂—, and R4 being an alkyl group, preferably a branched alkyl group such as the tert-butyl group
A1 is Val, Leu, or is absent
AspSubst, is an aspartic acid residue of formula IV

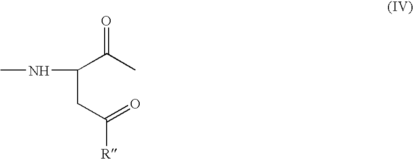

wherein R″ is
is O-alk, alk being a C1-C5 alkyl, or represents
“Linker-D”, with
“Linker” being —O— with one or several amino acids grafted thereon such as Gly or Gly-Phe-Leu-Gly-, or NH or NHCO, or CO—O—, or a malonyl group, and
“D” being
either a HPMA polymer (N-(2-hydroxypropyl)metheacrylamide polymer), or
Y, which represents a group of formula V

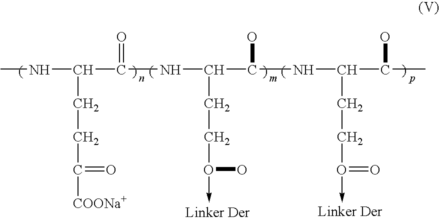

with n≧1; m≧1; p=0 or ≧1
wherein “Der” means a derivative of formula I,
or R″ represents Z which is —(O)ₙ-PEG (polyethylene glycol=PEG100-100000; n=0-1)
   or

### —(O)ₙ—(CO)ₘ—C(CH₃)H—NH—CO—CH₂O-PEG-X

with X═OH or OCH₂CO₂H and PEG (polyethylene glycol=PEG100-100000) and n=0-1 and m=0-1
or
—(O)ₙ—(CO)ₘ—CH₂—NW—CO—CH₂—O-PEG-X (polyethylene glycol=PEG100-100000); n=0-1; m=0-1; W═H or CH₃; with X═OH or OCH₂CO₂H)
or
—(O)ₙ—(CO)ₘ—CH₂—NW-PEG-X (polyethylene glycol=PEG100-100000); n=0-1; m=0-1; W═H or alkyl; with X═OH or OCH₂CO₂H)
or
—(O)ₙ—(CO)ₘ—CH₂—O-PEG-X (polyethylene glycol=PEG100-100000); n=0 or 1; m=0-1; X═OH or OCH₂CO₂H)
or
—(O)ₙ—(CO)ₘ—CH₂—O—CH₂—CO—NW-PEG-X (polyethylene glycol=PEG100-100000); n=0-1; m=0-1; X═OH or OCH₂CO₂H; W═H or alkyl)
or
Z1-Der wherein Z1 is —(O)ₙCO—C(CH₃)H—NH—CO—CH₂O-PEG-CH₂—CO—NH—C(CH₃)H—CO—(O)ₙ—
with PEG=PEG 100-100000; n=0-1
or
—(O)ₙ—(CO)ₘ—C(CH₃)H—NH—CO—CH₂O-PEG-OCH₂—CO—NH—C(CH₃)H—(CO)ₘ—(O)ₙ—
with X═OH or OCH₂CO₂H and PEG (polyethylene glycol=PEG100-100000), n=0-1 and m=0-1
or

### —(O)ₙ—(CO)ₘ—CH₂—NW—CO—CH₂—O-PEG-O—CH₂—CO—NW—CH₂—(CO)ₘ—(O)ₙ

with polyethylene glycol=PEG100-100000; n=0-1; m=0-1; W═H or CH₃; with X═OH or OCH₂CO₂H)
or

### —(O)ₙ—(CO)ₘ—CH₂—NW-PEG-NW—CH₂—(CO)ₘ—(O)ₙ

with polyethylene glycol=PEG100-100000; n=0-1; m=0-1; W═H or alkyl; with X═OH or OCH₂CO₂H
or
—(O)ₙ—(CO)ₘ—CH₂—O-PEG-O—CH₂—(CO)ₘ—(O)ₙ (polyethylene glycol=PEG100-100000); n=0-1; m=0-1; X═OH or OCH₂CO₂H)
or

### —(O)ₙ—(CO)ₘ—CH₂—O—CH₂—CO—NW-PEG-NW—CO—CH₂—O—CH₂—(CO)ₘ—(O)ₙ

(polyethylene glycol=PEG100-100000); n=0-1; m=0-1; X═OH or OCH₂CO₂H; W═H or alkyl)
and “Der” is as above defined.
or R″ represents J of formula VI

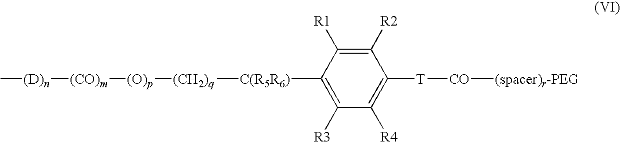

wherein

### D=O ou NH

n=0-1
m=0-1
p=0-1
q=0-1
i=0-1
r=0, 1-10
R1, R2, R3, R4=H or alkyl
R5, R6=H or alkyl
Spacer=one amino acid (for instance, alanine, proline, β-alanine, NH(CH₂CH₂O)₂, NH(CH₂CH₂O)CH₂CH₂NH

### T=O or NH

### PEG=PEG100-100000

A is
either A2-A3, with A2 being Val or Glu and A3 being Ala, Ser, Tic (1,2,3,4-tetrahydroisoquinoline-3-carbonyl) and Aic (2-amino-2,3-dihydro-1H-indene-2-carbony),
or
A2-A3 being 3-amino-4-oxo-1,2,3,4,6,7-hexahydroazepino[3,2,1-hi]indole-6-carbonyl,
R1 is selected in the group comprising —CH₂O—,
R2 is a phenyl group substituted by one or several groups, identical or different, selected amongst the halogen atoms and/or alkyl, alkoxy, carboxyl, 1-oxoalkyl groups and the pharmaceutically acceptable salts thereof.
   Said formula I covers all stereoisomers (diastereoisomers and enantiomers) and all racemic forms.

The abbreviation given herein above to designate the amino acid residues are those commonly used, i.e. Val=valine; Asp=aspartic acid, Ala=alanine, Glu=glutamic acid, Leu=leucine, Gly=glycine

As shown in the experimental results given hereinafter in the Examples, the above disclosed derivatives specifically prevent caspase-2 activation, thus preventing activation of downstream caspase-3.

The invention then provides means of great interest to treat any cardiac pathology involving caspase-2 activation as it occurs in myocardial ischemia.

Said derivatives of formula (I) are then useful for making drugs for treating cardiac pathologies resulting from myocardial ischemia.

In formula (I), A1 and A2 are advantageously a valine residue. Alternatively, A1 is a valine residue and A2 is a glutamic acid residue.

Preferably, Asp Subst in formula (I) is an aspartyl residue with R″ representing OCH₃ group.

The derivatives wherein R2 is a phenyl group substituted by 2 to 5 fluorine atoms correspond to particularly valuable active principles of drugs.

The invention particularly relates to the above use wherein the derivative is selected in the group comprising.

D1: (3 S,6 S,9 S,12S)-methyl 15-(2-(2,6-difluorophenoxy)acetyl)-3,9-diisopropyl-6-(2-methoxy-2-oxoethyl)-12-methyl-1,4,7,10,13-pentaoxo-1-(quinolin-2-yl)-2,5,8,11,14-pentaazaheptadecan-17-oate of formula VII

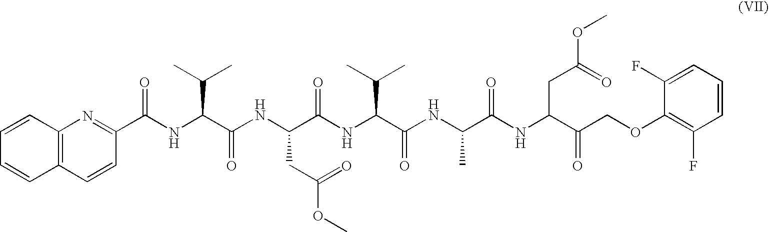

D2: methyl 5-(2,6-difluorophenoxy)-3-((S)-2-((S)-2-((S)-4-methoxy-2-4S)-3-methyl-2-(quinoline-2-carboxamido)butanamido)-4-oxobutanamido)-3-methylbutanoyl)-1,2,3,4-tetrahydroisoquinoline-3-carboxamido)-4-oxopentanoate of formula VIII

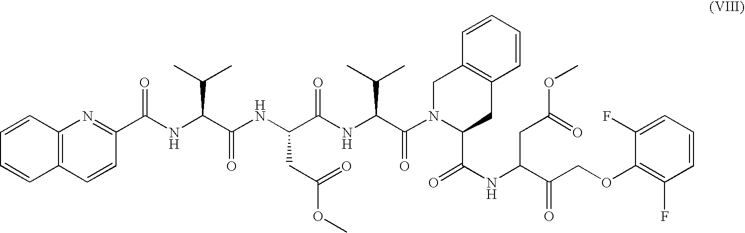

D3: methyl 5-(2,6-difluorophenoxy)-3-(2-((S)-2-((S)-4-methoxy-2-((S)-3-methyl-2-(quinoline-2-carboxamido)butanamido)-4-oxobutanamido)-3-methylbutanamido)-2,3-dihydro-1H-indene-2-carboxamido)-4-oxopentanoate of formula IX

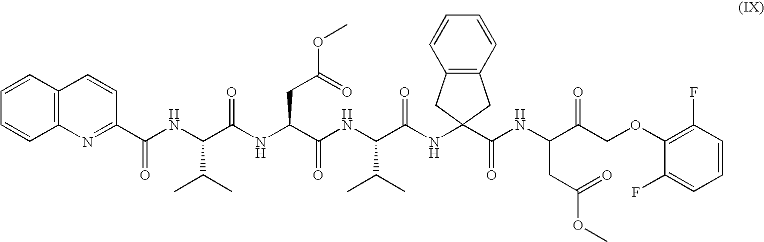

D4: (4S)-5-((2S)-1-(5-(2,6-difluorophenoxy)-1-methoxy-1,4-dioxopentan-3-ylamino)-1-oxopropan-2-ylamino)-4-((S)-4-methoxy-2-((S)-3-methyl-2-(quinoline-2-carboxamido)butanamido)-4-oxobutanamido)-5-oxopentanoic acid of formula X

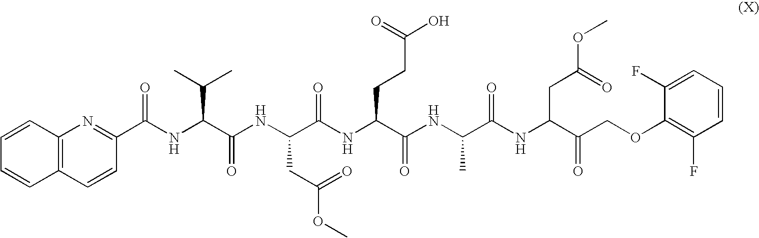

D5: (4S)-5-((2S)-1-(5-(2,6-difluorophenoxy)-1-methoxy-1,4-dioxopentan-3-ylamino)-3-hydroxy-1-oxopropan-2-ylamino)-4-((S)-4-methoxy-2-((S)-3-methyl-2-(quinoline-2-carboxamido)butanamido)-4-oxobutanamido)-5-oxopentanoic acid of formula XI

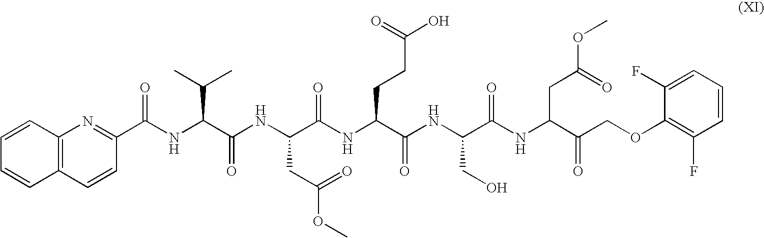

insofar as they are not under the form of salts.
Preferred derivatives used according to the method of the invention are selected in the group comprising:
D1: (3S,6S,9S,12S)-methyl 15-(2-(2,6-difluorophenoxy)acetyl)-3,9-diisopropyl-6-(2-methoxy-2-oxoethyl)-12-methyl-1,4,7,10,13-pentaoxo-1-(quinolin-2-yl)-2,5,8,11,14-pentaazaheptadecan-17-oate of formula VII

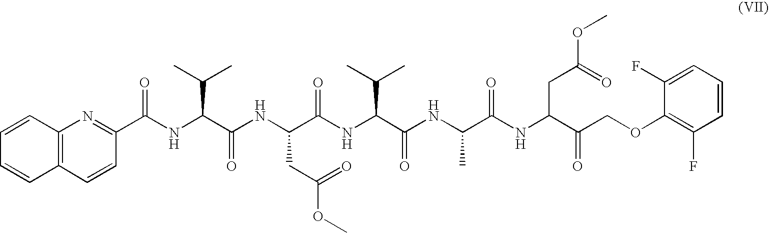

D2: methyl 5-(2,6-difluorophenoxy)-3-((S)-2-((S)-2-((S)-4-methoxy-2-((S)-3-methyl-2-(quino line-2-carboxamido)butanamido)-4-oxobutanamido)-3-methylbutanoyl)-1,2,3,4-tetrahydroisoquinoline-3-carboxamido)-4-oxopentanoate of formula VIII

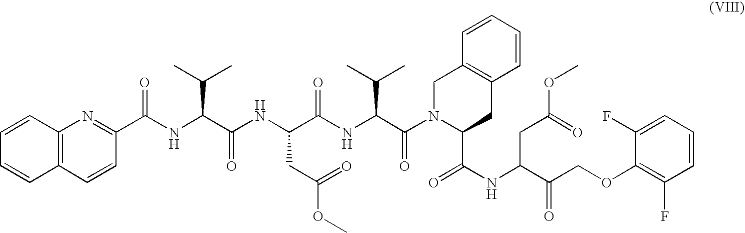

D3: methyl 5-(2,6-difluorophenoxy)-3-(2-((S)-2-((S)-4-methoxy-2-((S)-3-methyl-2-(quinoline-2-carboxamido)butanamido)-4-oxobutanamido)-3-methylbutanamido)-2,3-dihydro-1H-indene-2-carboxamido)-4-oxopentanoate of formula IX

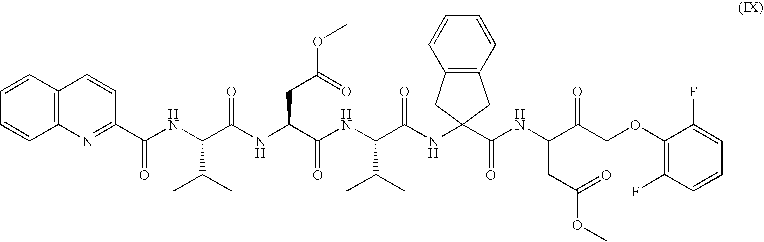

D4: (4S)-5-((2S)-1-(5-(2,6-difluorophenoxy)-1-methoxy-1,4-dioxopentan-3-ylamino)-1-oxopropan-2-ylamino)-4-((S)-4-methoxy-2-((S)-3-methyl-2-(quinoline-2-carboxamido)butanamido)-4-oxobutanamido)-5-oxopentanoic acid of formula X

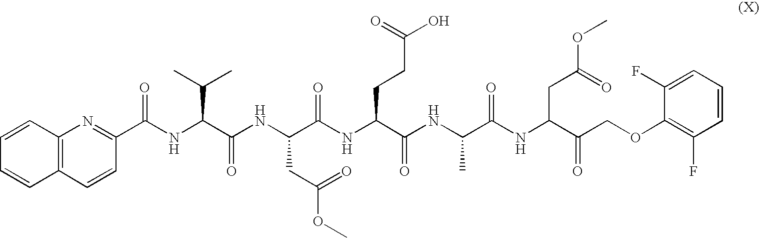

D5: (4S)-5-((2S)-1-(5-(2,6-difluorophenoxy)-1-methoxy-1,4-dioxopentan-3-ylamino)-3-hydroxy-1-oxopropan-2-ylamino)-4-((S)-4-methoxy-2-((S)-3-methyl-2-(quinoline-2-carboxamido)butanamido)-4-oxobutanamido)-5-oxopentanoic acid of formula XI

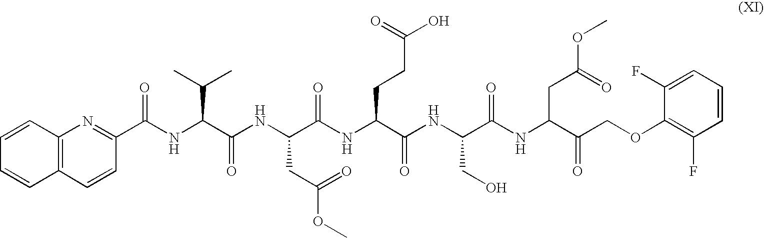

D6: (3 S,6 S,9 S,12 S)-methyl 3,9-diisopropyl-6-(2-methoxy-2-oxo ethyl)-12-methyl-1,4,7,10,13-pentaoxo-1-(quinolin-2-yl)-15-(2-(2,3,5,6-tetrafluorophenoxy)acetyl)-2,5,8,11,14-pentaazaheptadecan-17-oate of formula XII

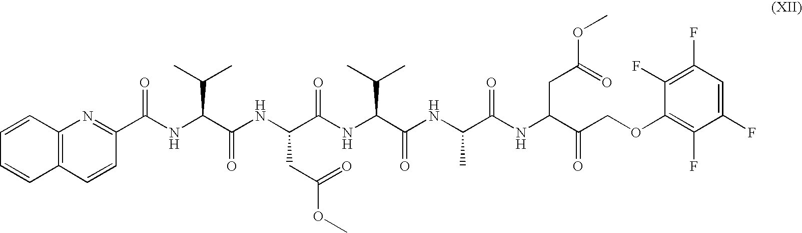

D7: methyl 5-(2,6-difluorophenoxy)-3-((3S,6S)-3-((S)-4-methoxy-2-((S)-3-methyl-2-(quinoline-2-carboxamido)butanamido)-4-oxobutanamido)-4-oxo-1,2,3,4,6,7-hexahydroazepino[3,2,1-hi]indole-6-carboxamido)-4-oxopentanoate of formula XIII

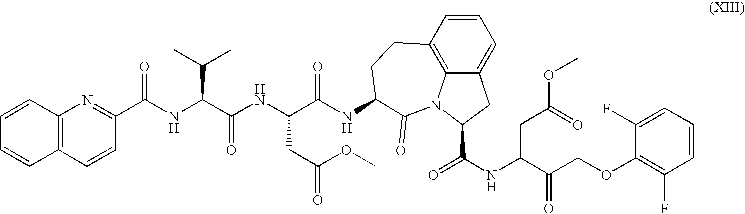

D8: (4S,7S,10S,13S)-methyl 1-(2-tert-butylphenylamino)-16-(2-(2,6-difluorophenoxy)acetyl)-4,10-diisopropyl-7-(2-methoxy-2-oxoethyl)-13-methyl-1,2,5,8,11,14-hexaoxo-3,6,9,12,15-pentaazaoctadecan-18-oate of formula XIV

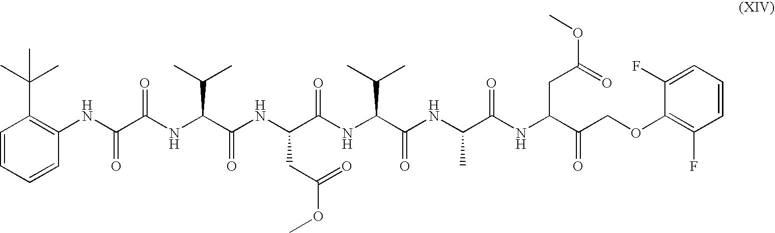

D9: (4S,7S,10 S,13S)-methyl 1-(2-tert-butylphenylamino)-4,10-diisopropyl-7-(2-methoxy-2-oxo ethyl)-13-methyl-1,2,5,8,11,14-hexaoxo-16-(2-(2,3,5,6-tetrafluorophenoxy)acetyl)-3,6,9,12,15-pentaazaoctadecan-18-oate of formula XV

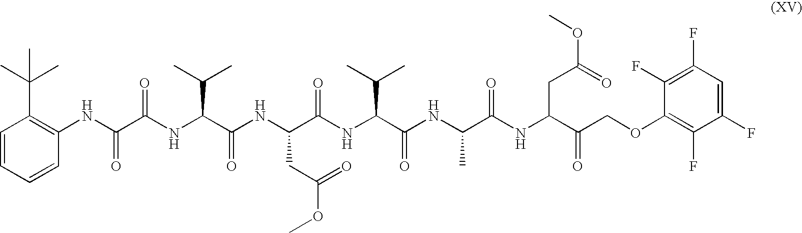

D10: (4 S,7 S,10 S)-methyl 1-(2-tert-butylphenylamino)-13-(2-(2,6-difluorophenoxy)acetyl)-7-isopropyl-4-(2-methoxy-2-oxo ethyl)-10-methyl-1,2,5,8,11-pentaoxo-3,6,9,12-tetraazapentadecan-15-oate of formula XVI

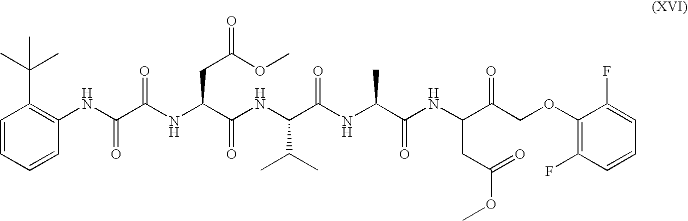

D11: (4 S,7 S,10S)-methyl 1-(2-tert-butylphenylamino)-7-isopropyl-4-(2-methoxy-2-oxo ethyl)-10-methyl-1,2,5,8,11-pentaoxo-13-(2-(2,3,5,6-tetrafluorophenoxy)acetyl)-3,6,9,12-tetraazapentadecan-15-oate of formula XVII

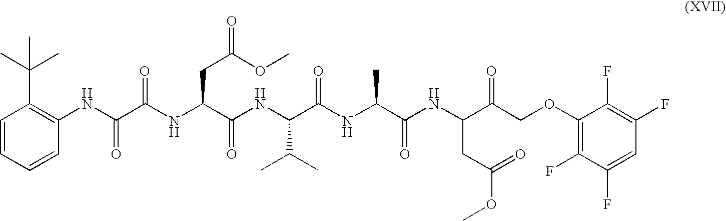

D12: (6S,9S,12S,15S)-methyl 19-(2-tert-butylphenylamino)-3-(2-(2,6-difluorophenoxy)acetyl)-9,15-diisopropyl-12-(2-methoxy-2-oxo ethyl)-6-methyl-5,8,11,14,17,19-hexaoxo-4,7,10,13,16-pentaazanonadecan-1-oate of formula XVIII

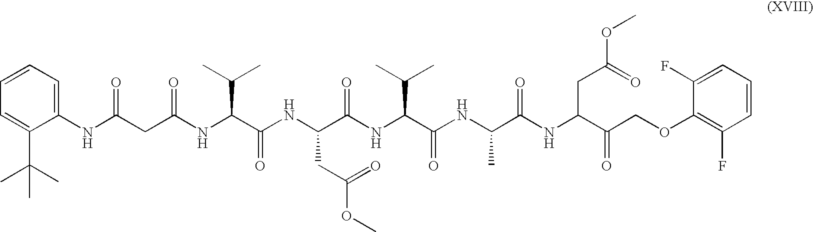

D13: (6 S,9 S,12 S,15S)-methyl 19-(2-tert-butylphenylamino)-9,15-diisopropyl-12-(2-methoxy-2-oxoethyl)-6-methyl-5,8,11,14,17,19-hexaoxo-3-(2-(2,3,5,6-tetrafluorophenoxy)acetyl)-4,7,10,13,16-pentaazanonadecan-1-oate of formula XIX

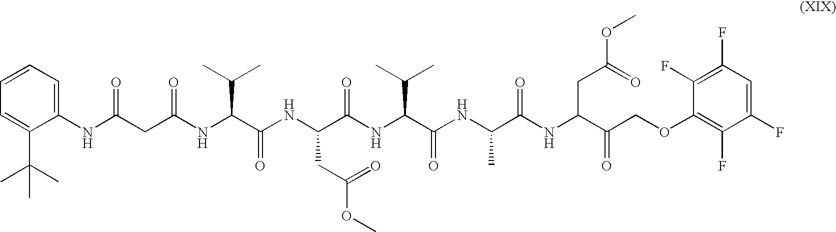

D14: (4S)-5-((2S)-1-(5-(2,6-difluorophenoxy)-1-methoxy-1,4-dioxopentan-3-ylamino)-1-oxopropan-2-ylamino)-4-((S)-4-methoxy-2-((S)-4-methyl-2-(quinoline-2-carboxamido)pentanamido)-4-oxobutanamido)-5-oxopentanoic acid of formula XX

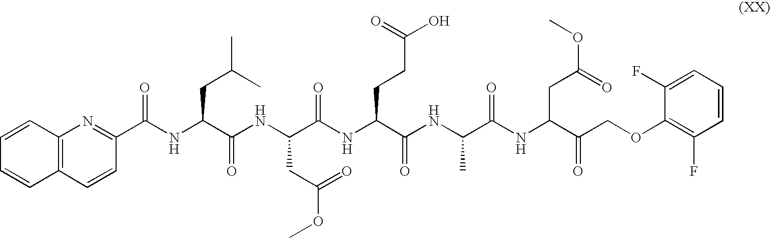

D15: (4S)-5-((2S)-1-(1-methoxy-1,4-dioxo-5-(2,3,5,6-tetrafluorophenoxy)pentan-3-ylamino)-1-oxopropan-2-ylamino)-4-((S)-4-methoxy-2-((S)-4-methyl-2-(quinoline-2-carboxamido)pentanamido)-4-oxobutanamido)-5-oxopentanoic acid of formula XXI

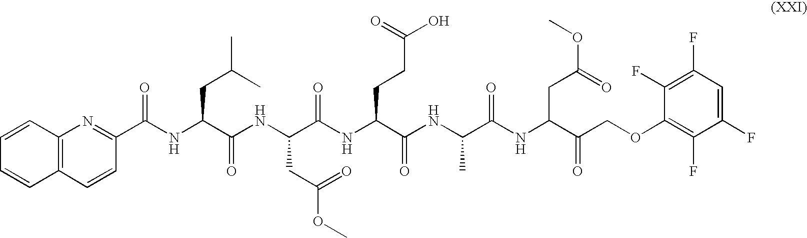

D16: N-(2-hydroxypropyl)methacrylamide copolymer-TRP601 (with A=D1), said derivative 18 having formula XXII

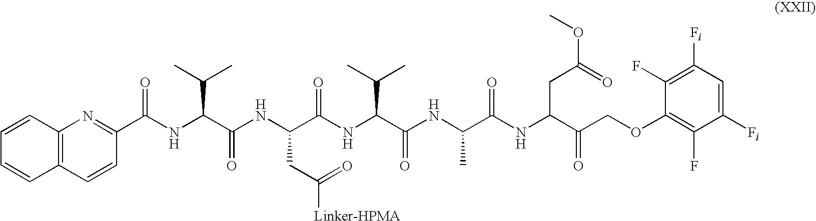

with
Linker=
one or several amino acids (Gly or Gly-Phe-Leu-Gly for example) grafted on the carboxylic function of the P4 Asp side-chain via an amide or ester function
a malonate derivative
i=0-1, with Fᵢ=H when i=0 and Fᵢ=F when Fᵢ=1
HPMA=N-(2-hydroxypropyl)methacrylamide polymer (n≧1; m≧1)

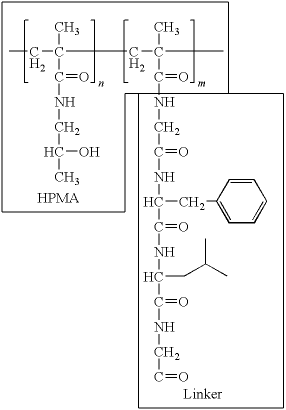

D17: Asp-Linker-Y polyglutamate-TRP601 (with A=D1), said derivative 19 having of formula XXIII

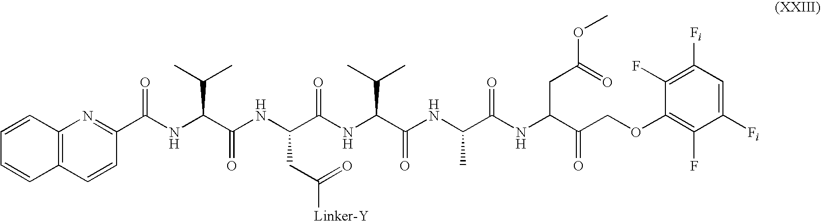

with
i=0-1, with Fᵢ=H when i=0 and Fᵢ=F when Fᵢ=1
Linker=one or several amino acids grafted on the COOH group of the P4 Asp side-chain.
Y=

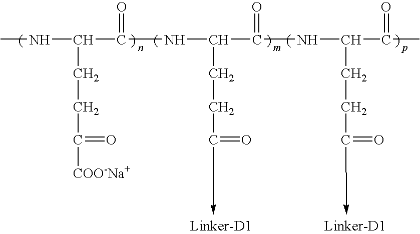

with n≧1; m≧1; p=0 or ≧1; i=0-1 and D1 is as above defined
D18: N^{α}-Quinoline-2-carbonyl-(S)-Val-(S)-Asp(Z)-(S)-Val-(S)-Ala-(R,S)-Asp(OMe)-CH₂O—C₆H₃-2,6-F₂ or N^{α}-Quinoline-2-carbonyl-(S)-Val-(S)-Asp(Z)-(S)-Val-(S)-Ala-(R,S)-Asp(OMe)-CH₂O—C₆H-2,3,5,6-F₄ of formula XXIV

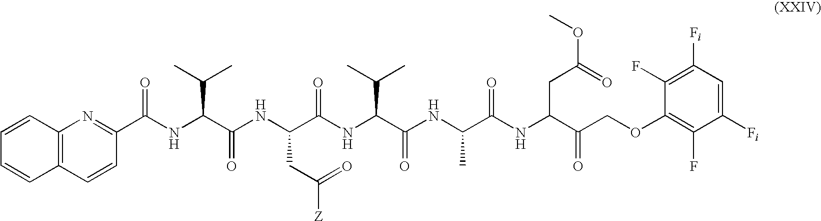

### Wherein:

i=0-1, with Fᵢ=H for i=0 and Fᵢ=F for i=1
and

Z=

—(O)ₙ—PEG (polyethylene glycol=PEG100-100000; n=0-1)
or

### —(O)ₙ—(CO)ₘ—C(CH₃)H—NH—CO—CH₂O-PEG-X

with X═OH or OCH₂CO₂H and PEG (polyethylene glycol=PEG100-100000) and n=0-1 and m=0-1
or
—(O)ₙ—(CO)ₘ—CH₂—NW—CO—CH₂—O-PEG-X (polyethylene glycol=PEG100-100000); n=0-1; m=0-1; W═H or CH₃; with X═OH or OCH₂CO₂H
or
—(O)ₙ—(CO)ₘ—CH₂—NW-PEG-X (polyethylene glycol=PEG100 à 100000); n=0-1; m=0-1; W═H or alkyl; with X═OH or OCH₂CO₂H
or
—(O)ₙ—(CO)ₘ—CH₂—O-PEG-X (polyethylene glycol=PEG100 à 100000); n=0 or 1; m=0-1; X═OH or OCH₂CO₂H
or
—(O)ₙ—(CO)ₘ—CH₂—O—CH₂—CO—NW-PEG-X (polyethylene glycol=PEG100-100000); n=0-1; m=0-1; X═OH or OCH₂CO₂H; W═H or CH₃ or alkyl
D19: TRP601-PEG-TRP601-(with A=D1), said derivative 21 having formula XXV

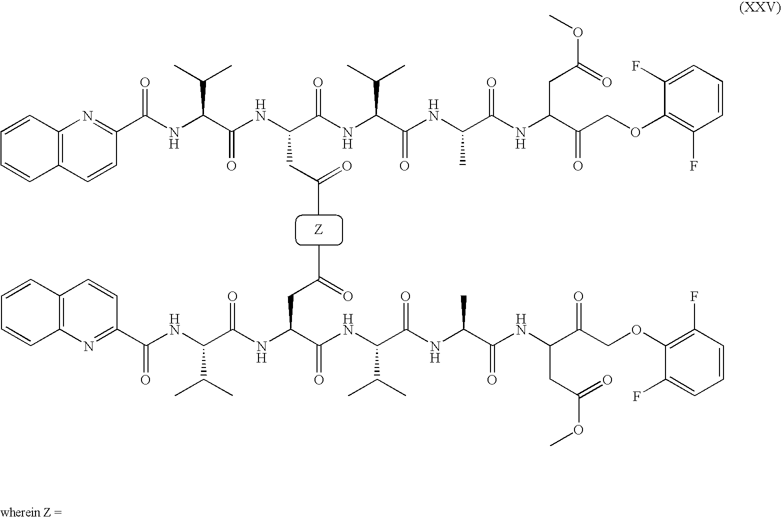

### —(O)ₙCO—C(CH₃)H—NH—CO—CH₂O-PEG-CH₂—CO—NH—C(CH₃)H—CO—(O)ₙ—

with PEG=PEG 100-100000; n=0-1
or
—(O)ₙ—(CO)ₘ—C(CH₃)H—NH—CO—CH₂O-PEG-OCH₂—CO—NH—C(CH₃)H—(CO)ₘ—(O)ₙ—
with X═OH or OCH₂CO₂H and PEG (polyethylene glycol=PEG100-100000); n=0-1 and m=0-1
or

### —(O)ₙ—(CO)ₘ—CH₂—NW—CO—CH₂—O-PEG-O—CH₂—CO—NW—CH₂—(CO)ₘ—(O)ₙ—

with polyethylene glycol=PEG100-100000; n=0-1; m=0-1; W═H or CH₃; with X═OH or OCH₂CO₂H)
or

### —(O)ₙ—(CO)ₘ—CH₂—NW-PEG-NW—CH₂—(CO)ₘ—(O)ₙ—

with polyethylene glycol=PEG100-100000; n=0-1; m=0-1; W═H or alkyl; with X═OH or OCH₂CO₂H
or
—(O)ₙ—(CO)ₘ—CH₂—O-PEG-O—CH₂—(CO)ₘ—(O)ₙ— (polyethylene glycol=PEG100-100000);
n=0-1; m=0-1; X═OH or OCH₂CO₂H
or

### —(O)ₙ—(CO)ₘ—CH₂—O—CH₂—CO—NW-PEG-NW—CO—CH₂—O—CH₂—(CO)ₘ—(O)ₙ—

(polyethylene glycol=PEG100-100000); n=0-1; m=0-1; X═OH or OCH₂CO₂H; W═H, CH₃ or alkyl
D20: N^{α}-Quinoline-2-carbonyl-(S)-Val-(S)-Asp(J)-(S)-Val-(S)-Ala-(R,S)-Asp(OMe)-CH₂O—C₆H₃-2,6-F₂ or N^{α}-Quinoline-2-carbonyl-(S)-Val-(S)-Asp(J)-(S)-Val-(S)-Ala-(R,S)-Asp(OMe)-CH₂O—C₆H-2,3,5,6-F₄ of formula XXVI

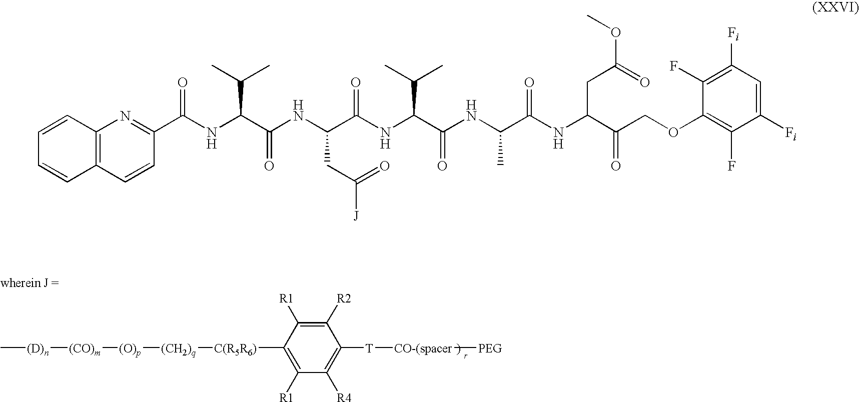

### D=O or NH

n=0-1
m=0-1
p=0-1
i=0-1, with Fᵢ=H for i=0 and Fᵢ=F for i=1
r=0, 1-10
R1, R2, R3, R4=H or alkyl
R5, R6=H or alkyl
Spacer=one amino acid (for example, alanine, proline, β-alanine, NH(CH₂CH₂O)₂,

### NH(CH₂CH₂O)CH₂CH₂NH

### T=O or NH

### PEG=PEG100-100000

Derivatives 6 to 20 are new compounds and are then specifically covered by the invention.

The invention also relates to the new derivatives of formula I for use as drugs.

The invention thus also concerns pharmaceutical compositions comprising therapeutically effective amount of at least one compound of formula I such as above defined except D1 to D5, in association with a pharmaceutically acceptable vehicle.

During the production of the drugs, the active ingredients, used in therapeutically effective amounts are mixed with the pharmaceutically acceptable vehicles for the mode of administration chosen. These vehicles may be solids or liquids or gels.

The drugs may be under a form suitable for an administration preferably by intravenous route, but also by oral or injectable route intramuscular and subcutaneous routes, or nasal route.

Thus, for administration by the oral route, the medicaments may be prepared in the form of gelatin capsules, tablets, sugar-coated tablets, capsules, pills and the like. Such medicaments may contain from 10 micrograms to 1 g of active ingredient per unit.

For administration by injection (bolus or perfusion; intravenous, subcutaneous, intraperitoneal, intratechal, intradermous), the medicaments are provided in the form of sterile or sterilizable solutions.

They may also be in the form of emulsions or suspensions.

The doses per dosage unit may vary from 1 micrograms to 1 g of active ingredient.

The caspase-2 inhibitors used according to the invention are particularly useful as therapeutical agents to reduce lesions and functional consequences of ischemic situations at the myocardium level, such as myocardium infarct, and other ischemic cardiopathies such as coronary cardiopathies, cardiac insufficiencies as well as septic shock, myocardites. They are generally useful for treating any processus having a strong inflammatory component or oxidative stress component. Said inhibitors are particularly useful for treatments at the brain level in adults and in neonates (global or focal cerebral ischemia, asphyxia, hypoxia-ischemia, traumatic brain injury), or in the eye, internal ear, kidney. These injuries and their duration may be transient or permanent. The above defined caspase-2 inhibitors are also of great value for the protection of grafts during heart, liver, skin and kidney transplant.

Other characteristics and advantages of the invention will be given in the results reported below in order to illustrate the invention.

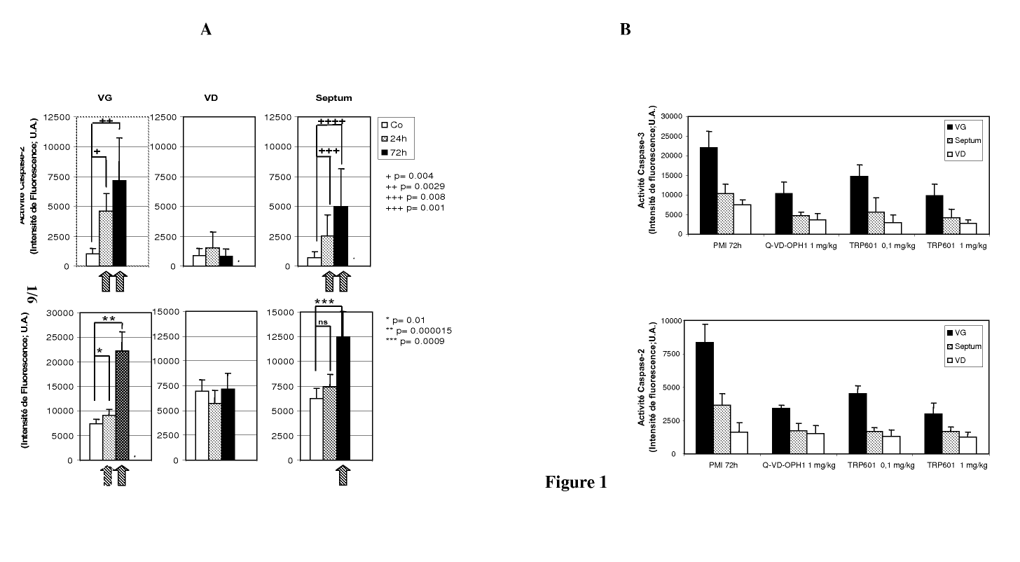

In these examples, reference is made to FIGS. 1 to 10, which represent, respectively,

FIG. 1: effect of caspase 2-specific inhibition by a derivative according to the invention compared to the effect of a pan-caspase inhibitor in rat chronic PMI (post myocardial infarction) model on caspase-2 (C2) and caspase-3 (C3) activities in left ventricle (VG), right ventricle (VD) and septum,

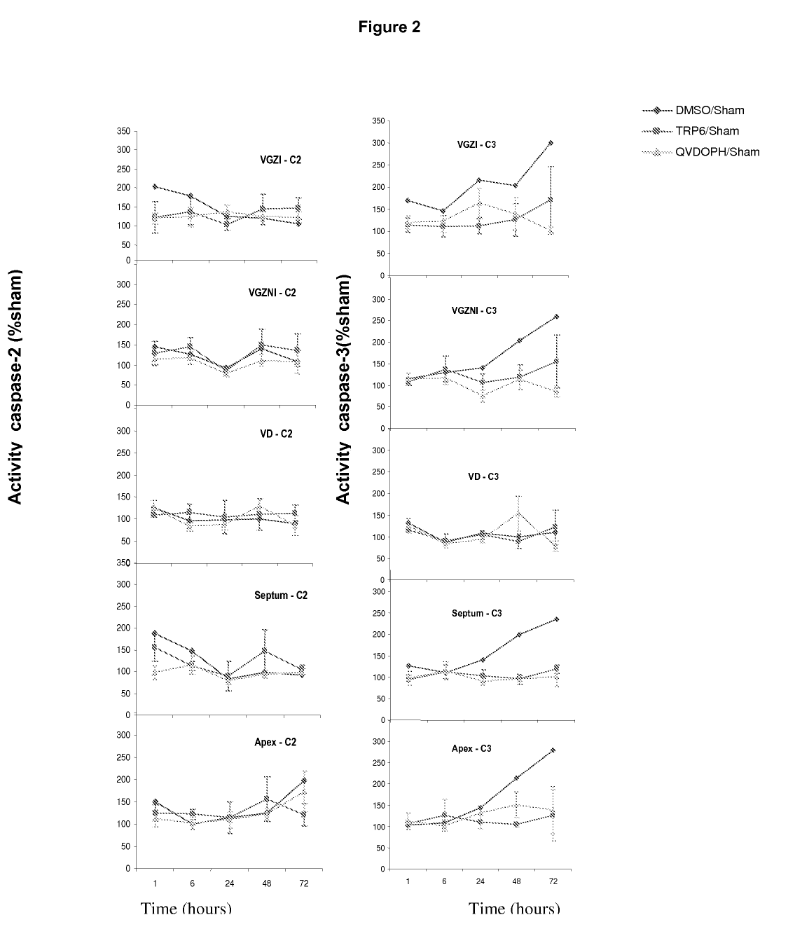

FIG. 2: Kinetics of caspase 2 (C2) and caspase 3 (C3) activation in left ventricle (selectively in infracted area (VGZI) and non-infarcted area (VGZNI)), right ventricle (VD), apex and septum before and after treatment by a derivative according to the invention or a pan-caspase inhibitor in myocardial ischemia-reperfusion model;

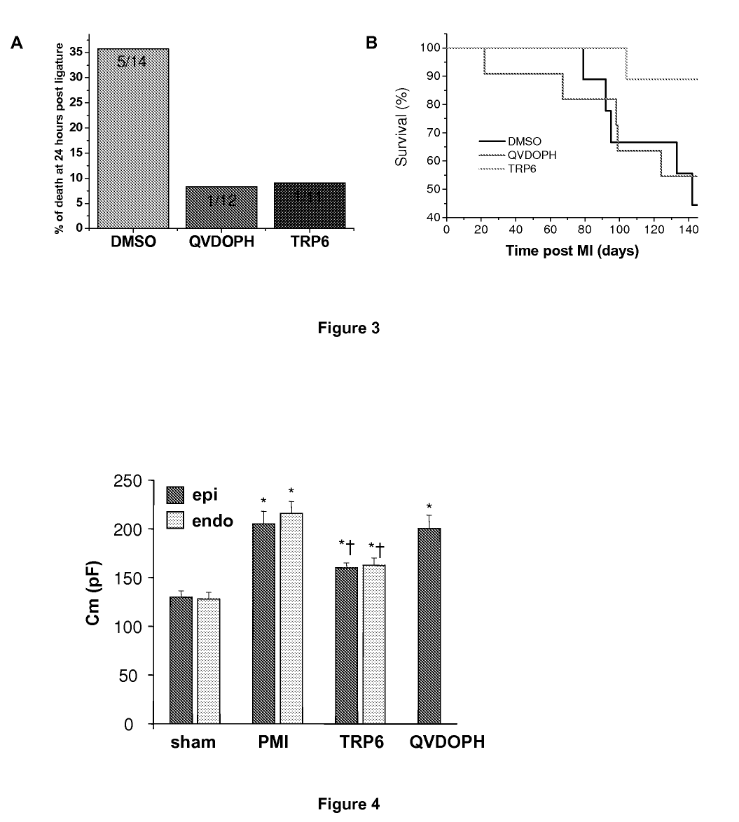

FIG. 3: the effect of a caspase 2-specific inhibitor (a derivative according to the invention) on animal survival after PMI.

FIG. 4: electrophysiological results relating to the prevention of the membrane capacitence when treating models with a pan-caspase inhibitor or a caspase 2-specific inhibitor;

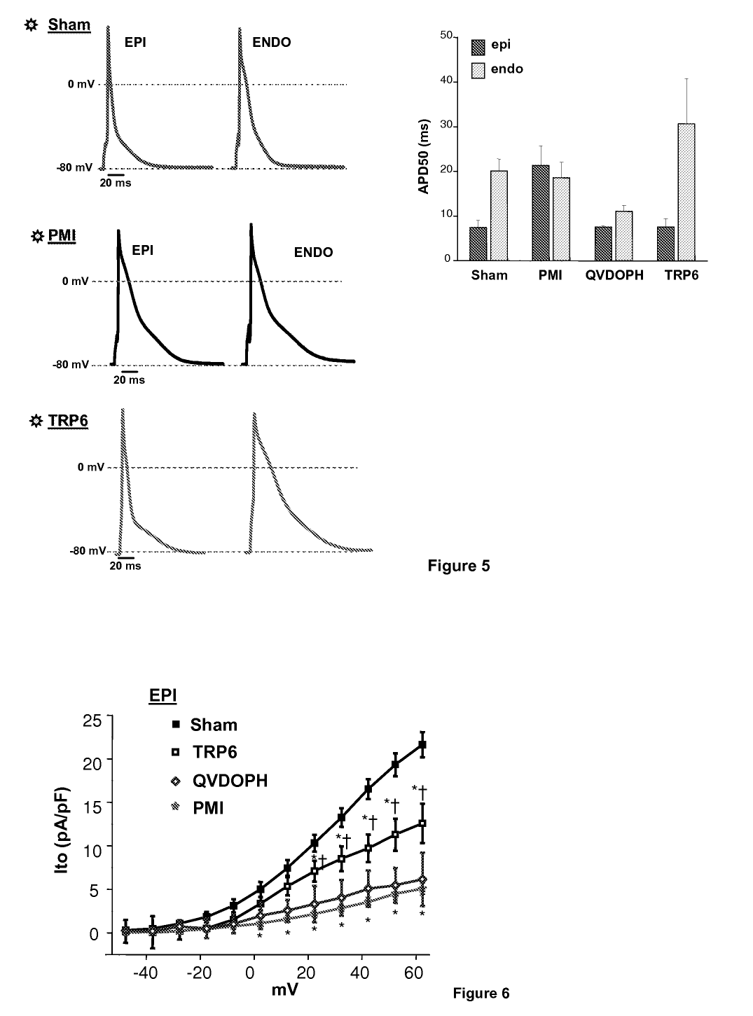

FIG. 5: results concerning potential of action registered on cardiomyocytes from endocardial and epicardial layers

FIG. 6: the relation between the density of current obtained with cardiomyocytes as a function of Ito (transitory current coming out);

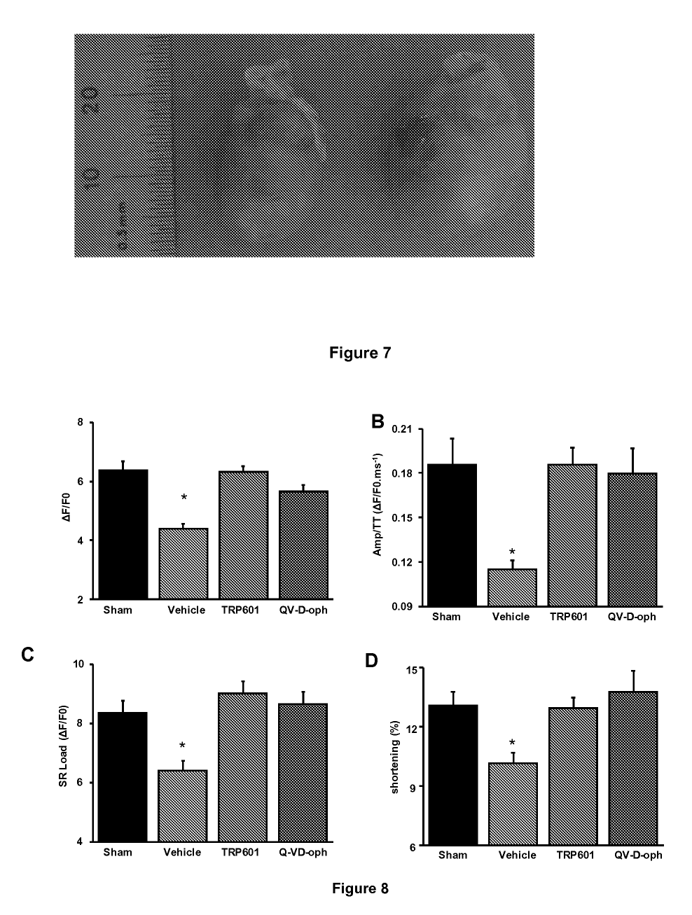

FIG. 7: the effect of a caspase 2-specific inhibitor on the cardiac hypertrophy,

FIG. 8: Ca²⁺ handling remodelling prevention by caspase-2 inhibition,

FIG. 9: acute inflammation response prevention by caspase-2 inhibition,

FIG. 10: left ventricular inflammation and remodelling prevention by caspase-2 inhibition.

### METHODOLOGY

### Models of Myocardial Ischemia

The analyses were performed on 2 myocardial ischemia models: a chronic ischemia model and an acute ischemia model, followed by myocardial reperfusion after 30 min of occlusion.

Said models were realized with Wistar male rats (180-220 g). The coronary artery was ligatured according to Pfeffer et al protocol.

Briefly, the rats were anaesthetized by intraperitoneal administration of a mixture of Ketamine (150 mg/kg) and Xylazine (15 mg/kg), then intubated and mechanically ventilated. The animals were submitted to a left and median thoracotomy. An occlusion of the coronary artery was done with a silk wire (size: 7.0) at the more proximal point and below the auricle.

In the case of chronic ischemia, the ligature was maintained and the rib cage of the animal reclosed (PMI model for post myocardial infarction).

In the case of reperfusion ischemia model (IR model), the silk wire was untied after 30 min of occlusion. The artery was then cleared and the myocardial territory reperfused. The control animals (sham operated) were submitted to the same surgery protocol as the above animals, but the silk wire was passed under the coronary without making any ligature.

### Dosage of the Caspase Activities

The animals were sacrificed by a pentobarbital lethal injection. The heart was excised and perfused about 5 min by Langerdorf reverse way using a calcium-free washing solution (in mM: NaCl 117 mM, KCl 5.7, NaHCO₃ 4.4, KH₂PO₄ 1.5, MgCl₂ 1.7, HEPES 21, glucose 11, taurine 20, pH 7.2 adjusted with NaOH). The heart was then placed into a dissection tank and the different myocardial territories were taken (i.e. right ventricle (VD), septum, Apex, left ventricle (selectively in infarcted area (VGZI) and non-infarcted area (VGZNI)). Each fragment of tissue was rapidly frozen in liquid nitrogen and kept at −80° C.

Each tissue was then thawed and about 1-2 mm³ was taken. The fragment was cut in small pieces which were put in a glass tube containing 500-800 μl of extraction buffer (buffer HEPES 10 mM pH 7.4, KCl 42 mM, MgCl₂ 5 mM, DTT 1 mM, CHAPS 0.5%, EDTA 0.1 mM; extemporeanously supplemented with protease inhibitors: PMSF 1 mM, leupeptin 1 μg/ml, pepstatin A 1 μg/ml, cytochalasin B 1 μM, chymopapain 10 μg/ml, antipain 1 μg/ml).

The tube was placed in a ice-bath and a mechanical crushing was performed. The crushed tissues were then transferred in an Eppenddorf of 1.5 ml, which was kept 24 h at −80° C. at least, in waiting for the elimination of the cellular remains.

Samples were thawned and put in a centrifugal machine (10 min, 4° C., 2000 g).

The supernatant was taken and divided into 2 tubes for of each sample. The tubes were placed at −80° C. before performing the spectrophotometric proteic dosage (BCA: cupper (II) sulphate+solution A of bicinchronic acid, DO measured at 550 nm) in transparent, with flat bottom, 96-well plates.

The dosage of the caspase activities was performed on black 96-well microplates with transparent and flat bottom.

100 μg of samples were diluted in a caspase activity buffer (Hepes 50 mM pH 7.4, NaCl 100 mM, DTT 10 mM, CHAPS 0.1%, EDTA 1 mM) to a final volume of 90 μl.

10 μl of caspase 2 (Ac-VDVAD-AMC) or caspase 3 (Ac-DEVD-AMC) specific substrate were added. The plate was incubated at 37° C. during 2-3 h in the dark. The measure was carried out by spectrofluorimetry (λex=380 nm; λem=460 nm).

### Histological Analyses

The animals were sacrificed by a lethal injection of pentobarbital. The heart was excised and perfused 2-3 min. using Langendorf reverse route and a calcium-free washing solution (NaCl 117, KCl 5.7, NaHCO₃ 4.4, KH₂PO₄ 1.5, MgCl₂ 1.7, HEPES 21, glucose 11, taurine 20, pH 7.2 adjusted to NaOH). The solution was then replaced by a PBS solution at 4% of PFA (about 10 ml). The heart was immersed in this fixation solution for about 1 h, and then washed with PBS 4%.

### Functional Analyses

### Isolation of Ventricular Cardiomyocytes

The isolated ventricular cardiomyocytes were obtained by enzymatic dissociation with collagenase by Langendorf reversed perfusion (Fauconnier, 2005). The rats were heparinized (0.2 ml, GIBCO® 1000 Ul/ml) and anaesthetized by intraperitoneal injection of pentobarbital (200 mg/100 g, Sanofi Santé, France). The heart was rapidly excised and a retrograde perfusion through the aorta, was performed for 5 min with a calcium-free washing solution (in mM: NaCl 117, KCl 5.7, NaHCO₃ 4.4, KH₂PO₄ 1.5, MgCl₂ 1.7, HEPES 21, glucose 11, taurine 20, pH 7.2 (adjusted with NaOH) and O₂-bubbled) at 37° C. The solution was then replaced by a similar medium containing 1.3 mg·ml⁻¹ of collagenase of type IV (Worthington, Freehold, N.J., USA) and perfused during 20-30 min. The heart was then perfused with the initial solution containing 2,3-butanedione monoxime as inhibitor of the muscular contraction (15 mM BDM). The ventricles were then delicately separated and, by mechanic stirring, the cardiomyocytes were liberated in the medium. The dissociated cells were washed in the same solution wherein increased concentrations of CaCl₂ were added (0.3, 1, 1.8 mM). The cells of the sub-epicardial layer (EPI) were separated from the sub-endocardial layer (ENDO) by simple manual dissection.

### Electrophysiological Recordings

The potentials of action (PA) and ionic currents were measured by the patch-clamp technique in whole cell configuration using an amplifier RK 400 (Biologic, Claix France) interfaced by a analogical/numerical converter DIGIDATA 1200 (Axon Instrument, Sunnyvale, Calif., USA) controlled by a PC. The acquisition and analysis of the data were realized with pCLAMP program (Axon Instrument, Sunnyvale, Calif., USA). The sampling frequency was of 10 KHz and the signals filtrated at 3 KHz. Pipettes comprised between 1 and 1.5 MΩ were used to ensure a good quality of voltage. For the measure of the potentials of action, the pipettes were filled with an internal solution (in mM: 130 KCl, 25 HEPES, 3 MgATP, 0.4 NaGTP, and 0.5 EGTA; the pH was adjusted at 7.2 with KOH). The external medium was composed of (in mM): 135 NaCl, 1 MgCl₂, 4 KCl, 11 glucose, 2 HEPES, and 1.8 CaCl₂; the pH was adjusted to 7.2. with NaOH.

The PA were started by injections of current of 0.2 ms at an intensity slightly higher than the supraliminal intensity threshold. The transitory potassium current coming out (Ito) was measured with the same internal and external solution (10 μM of tetrodotoxine (TTX) and 2 mM of cobalt chloride were added to the external medium to become independent from the potassic and calcic currents, respectively. Ito was measured from depolarizing pulses.

### Results

### Caspase Activities

The dosage of caspases-2 and -3 activities was performed at different points of the myocardium on both myocardial ischemia models. The results are given on FIGS. 1 (PMI model) and 2 (reperfusion ischemia).

On FIG. 1 (A), left ventricular (LV) and septum were been identified as being the cardiac tissues having a significant increase in caspase-2 and caspase-3 activity activation with an earlier of caspase-2.

In FIG. 1 (B), the rats having undergone a PMI during 72 h have been pre-treated by Q-VD-OPH (pan-caspase inhibitor) and D1 (selective and irreversible inhibitor of caspase-2).

At 72 h, the caspase-2 activity was inhibited by both derivatives in the left ventricle (VG) and the septum: D1 and Q-VD-OPH have comparable effects at 1 mg/kg. Caspase-2 inhibition by D1 (0.01 or 1 mg/kg) results in an inhibition of the caspase-3 activity showing that caspase-3 activation is strongly dependent on caspase-2. A weak inhibition of caspase-2 and caspase-3 basal activities was also observed in the right ventricular (RV).

The kinetics of caspase-2 and caspase-3 activations are given on FIG. 2.

Caspase-2: VGZI, Septum: early activation 1 h after reperfusion, then decrease of the activity up to 24 h and normalization of the activity with respect to the sham group.

VGZNI, VD, Apex: absence of activation, basic activity

Caspase3: VGZI, VD, Apex and VGZNI: secondary activation fro 6 h after reperfusion and at least up to 72 h. VD: absence of activation of C3 activity, basic activity.

Inhibition of the caspase-2 and caspase-3 activities by D1 and QVDOPH (administered 15 min before the reperfusion):

Caspase-2: VGZI, Septum: equivalent inhibition for both inhibitors, normalization of the activity.

Caspase-3: VGZI, VD, Apex and VGZNI: equivalent inhibition for both inhibitors, normalization of the activity.

The use of a broad spectrum caspase inhibitor, Q-VD-OPH, inhibits the activity of both caspases. D1 specifically inhibits caspase-2.

Interestingly, D1 prevents the further activation of caspase-3, the activation of which appears to be dependent on caspase-2 activation.

This experiment demonstrates for the first time that the activation of caspase-2 is transitory and early during the ischemic episode. Its activation appears to be a pre-requirement for the installation of apoptosis in heart.

Survival of Animals after Chronic Ischemia (PMI)

The effect of the blocking of the caspase activity by Q-VD-OPH or by D1 on animals survival 24 h after infarction are given on FIG. 3. (A) over a period of 140 days; (B) the injection of the inhibitors or vehicle (DMSO) was carried out by intraperitoneal route 5-15 min before the occlusion of the coronary artery.

The animals having undergone a permanent ligature of the left coronary artery (PMI) were maintained alive during 4-5 months before performing the in vivo and ex vivo functional exploration. The death of the animals during this period was followed up.

During the early phase (<24 hours) after the coronary ligature, a significant number of deaths was classically registered on the groups of operated animals. 5 animals on 14 early dye (35%), mainly because of severe rhythm disorders resulting from the artery occlusion.

The animals treated either by a broad caspase inhibitor (Q-VD-OPH), or by a caspase-2 specific inhibitor have a significant decrease of early death (<10%). In this situation, the specific inhibitor appears to have an effect comparable to the one of broad spectrum inhibitor.

The remaining animals were maintained alive over a period of about 140 days. In PMI animals, stable survival up to about 80 days was observed (date at which the animals regularly died to reach a survival of about 45% at 140 days).

Surprisingly, in the animals group treated with Q-VD-OPH, deaths of several animals were noticed from the 20^{th} day, then the survival curve superimposes on the one of the PMI group.

On the contrary, with the animals treated with D1, only 1 animal on 10 died within the 140 days post-infarctus, and this at 110 days. These results clearly demonstrate the benefic effect of caspase-2 inhibition on the animals survival over a long post-ischemic duration. Such an effect is never observed with a broad spectrum inhibitor which on the contrary results in deaths earlier than in absence of treatment.

### Morpho-Functional Analyses

### Model of Chronic Ischemia (PMI)

On the functional level, the animals subject to chronic ischemia (PMI) undergone two echocardiographies, the first one after 1 month and the second one after 4 months of infarct.

The morpho-functional data thus obtained did not reveal any clear benefic effect on the specific or not specific inhibitions of the caspases. After the 2^{nd} echocardiography, the animals were sacrificed before carrying out an electrophysiological cellular analysis.

The patch-clamp electrophysiological technique enables the membrane capacity (which reflects the cellular size) to be measured (this capacity reflects the cellular size). The results obtained with the above animals groups confirm the hypertrophy induced by the myocardial ischemia. Surprisingly, this hypertrophy is highly prevented by pre-treatment with D1. On the contrary, the treatment with a broad spectrum caspase inhibitor has not effect on the hypertrophic cellular remodeling (FIG. 4).

In the normal conditions, the potential of action is heterogeneous in the whole myocardial region. The duration of the potential of action is shorter in sub-endocardic layers than in sub-epicardic layers. The heterogeneity, mainly related to the one of the transitory K+ current coming out (Ito) which re-polarizes the PA at the initial phase of the plate tends to become standardized during ischemic pathology as previously described (Aimond 1999).

FIG. 5 gives the potentials of action recorded on cardiomyocytes of sub-epicardial and sub-endocardial layers of sham, PMI and TRP601-PMI treated rats.

ADP50 corresponds to the duration of the potential of action measured at 50% of its repolarization. This index allows quantification of the heterogeneity of the duration of PA between EPI and ENDO. It rationally demonstrates the extension of PA in PMI essentially in EPI.

In a remarkable way, on the PMI rats treated will D1 a total absence of PA remodeling is observed with the maintenance of the transmural (across the left ventricular free wall) gradient PA duration, characteristic of a normal electrical activity (no modification of the duration of the potential of action and maintenance of the transmural heterogeneity) after a pre-treatment of the animals by caspase 2 inhibitor (FIG. 5).

On FIG. 6, are given the results obtained when measuring sham animals and PMI animals injected which, DMSO, Q-VD-OPH and D1. The current/potential relation gives the results obtained on cardiomyocytes of the sub-epicardic layer. An important decrease of the current density is observed with the PMI which are very preserved by a treatment with D1 while the animals treated by Q-VD-OPH do not present any improvement of the current compared to PMI.

These results are essentially linked to the Ito decrease observed in the PMI animals which is significantly restored in the animals treated by D1 while Q-VD-OPH has no effect on the density of the current (FIG. 6).

### Re-Perfusion Ischemia Model

The animals were submitted to an occlusion of the left coronary artery during 30 min.

15 min before the re-perfusion, the animals were treated by an IP injection of D1 or DMSO. The animals were sacrificed after 72 h, 10 days after re-perfusion for an histological analysis of the hearts. The morphology of the hearts (PFA fixed 4%) 10 days after infarct is illustrated by FIG. 7.

The ischemiated and re-perfused hearts have a size significantly higher than the one of the animals treated by D1 (on the left). These results demonstrate the specific inhibition of caspase-2 in this model prevents cardiac hypertrophy.

### Evaluation of Functional Consequences of Caspase 2 Inhibition:

Experiments were carried out on single ventricular cardiomyocytes isolated from rats after 30 minutes of myocardial ischemia followed by 10 days of reperfusion. Cardiomyocytes were loaded with a fluorescent calcium indicator (Fluo4) in order to measure calcium transient evoked by 0.5 Hz field stimulation. Cell shortening was also simultaneously recorded as an index of cardiomyocyte contraction. The results are given on FIG. 8: A. Amplitudes of cytosolic Ca²⁺ transients measured under confocal microscopy were significantly decreased when only the vehicle was injected prior reperfusion. These deleterious effects classically reported by others in similar experimental conditions Under TRP601 or QV-D-oph treatment Ca²⁺ transients amplitude was unchanged B. Similarly, Ca²⁺ release kinetics estimated by the amplitude/time to peak (TT) ratio was slowed down in cardiomyocytes of untreated animals, whereas under TRP601 or QV-D-oph treatment Ca²⁺ release was unaffected. C. Altered Ca²⁺ transient kinetics in vehicle-treated animal are accompanied by a decrease in sarcoplasmic reticulum Ca²⁺ load (estimated by application of 10 mM caffeine). SR Ca²⁺ load in sham-operated, TRP601 or QV-D-oph animals were not significantly different. D. As well as Ca²⁺ transients' kinetics, cell shortening, an estimation of cardiomyocytes contraction, was significantly decreased in vehicle animals compared to sham-operated animal. TRP601 or QV-D-oph avoided cell shortening decreasing. In conclusion, after 30 min of ischemia followed by 10 days of reperfusion, caspase-2 activation contributes to functional cardiomyocytes remodelling. (*p<0.05 compared to sham-operated animals, n≧5 animals).

These data demonstrate the early protective effect of caspase 2 inhibition on functional physiopathological remodeling of the heart after ischemia and reperfusion.

Another series of experiments was conducted in the rat model of ischemia-reperfusion. Ischemia and reperfusion is well known to trigger an acute inflammatory process in animal model as well as in human. In our rat model, at the euthanasia, blood sample were collected in order to measure the inflammatory response on sera. This was performed by multiplex assay (Luminex technology). Three parameters were simultaneously measured at various time after myocardial reperfusion the pro inflammatory cytokines TNFα and IL1β as well as the anti-inflammatory cytokine IL10. The results are given on FIG. 9: A. Circulating level of TNFα was significantly elevated in vehicle-injected animal within the first hour after reperfusion and return to normal level after 6 h. When TRP601 or Q-VD-oph was injected 15 min prior to the reperfusion, circulating level of TNFα was not significantly different compare to sham-operated animal. B-C. Within the first 24 hours of reperfusion, in non-treated animals, peak TNFα level was tightly followed by a significant increase in IL-1β and IL-10, another pro-inflammatory and an anti-inflammatory cytokine respectively. TRP601 and QV-D-oph treatment also prevented IL-1β and IL-10 elevation. In summary, caspase 2 inhibition prevents the acute inflammatory response occurring during the first 24 hours after myocardial reperfusion. *p<0.05 compared to sham-operated animals, n≧5 animals.

It can be concluded from this observation that early activation of caspase 2 following myocardial infarction and reperfusion is a key trigger of the acute inflammatory response initially represented by TNFα.

In order to determine the potential physiopathological importance of TNFα the effect of Etanercept was also explored in addition to caspase inhibitors. Etanercept is a recombinant fusion protein encoding for the human soluble TNF receptor linked to the Fc component of human immunoglobulin G1 (IgG1), that binds to TNFα and decreases its role in disorders mediated by excess TNFα.

In a second series of experiments, quantitative RT-PCR was performed to evaluate the level of expression of 2 markers of heart failure, Atrial Natriuretic Factor (ANF) and Brain Natriuretic Factor (BNP) as well as a marker of myocardial fibrosis, fibronectine, and myocardial hypertrophy, βMHC. The results are given on FIG. 10: A. mRNA expression level of Atrial Natriuretic Factor (ANF) and brain natriuretic peptide (BNP), two markers of heart failure, were significantly increased in vehicle-treated animal. In TRP601- or QV-D-oph-treated rats mRNA expression levels of both markers were comparable to sham-operated animals. Etanercept-treated animal presented also a significant increase in ANF and BNP expression level. B. mRNA expression levels of fibronectin a marker of fibrosis development, and β-Myosin Heavy Chain (β-MHC) a marker for hypertrophy development

Both marker were significantly increased in vehicle-treated animal and blocked by TRP601 or QV-D-oph treatement. However, Ethanercept treatment could only prevent fibrosis development but not the level of hypertrophic marker. C. mRNA expressions levels of TNFα receptor 1 & 2 (TNFR1, TNFR2), TNFα, caspase-2 were significantly increase in vehicle-injected animal. This was prevented by TRP601 or QV-D-oph but not by Etanercept treatment. All together, these results show that caspase-2 inhibition prevents heart failure development as well as fibrosis and hypertrophy whereas Etanercept was unable to prevent hypertrophy and heart failure. In parallel, caspase-2 inhibition avoids up-regulation of TNFα signaling pathway whereas Etanercept did not, indicating that caspase-2 activation initiate inflammatory response and left ventricular remodeling after ischemia/reperfusion. *p<0.05 compared to sham-operated animals; #p<0.05 compared to vehicle-injected animals; n≧5 animals.

From these observations, it was concluded that caspase 2 activation after IR is a key trigger in the myocardial remodelling and inflammatory response. Nevertheless, TNFα can also act as a physiopathological actor secondary to caspase 2 activation and appears to amplify the physiopathological processes in the development of heart failure partially and fibrosis but without any effect in the hypertrophic response of the myocardium.

### BIBLIOGRAPHIC REFERENCES

Pfeffer M A, Pfeffer J M, Fishbein M C, Fletcher P J, Spadaro J, Kloner R A, Braunwald E. (1979) Myocardial infarct size and ventricular function in rats. Circ Res. 44(4):503-12.
Fauconnier J, Lacampagne A, Rauzier J M, et al. Ca2+-dependent reduction of IK1 in rat ventricular cells: a novel paradigm for arrhythmia in heart failure? (2005) Cardiovasc Res.; 68(2):204-212.
Aimond F, Alvarez J L, Rauzier J M, et al. Ionic basis of ventricular arrhythmias in remodeled rat heart during long-term myocardial infarction. Cardiovasc Res. May 1999; 42(2):402-415.
Mocanu M M, Baxter G F, Yellon D M. Caspase inhibition and limitation of myocardial infarct size: protection against lethal reperfusion injury.
Br J. Pharmacol. 2000 May; 130(2):197-200

1- Use of a caspase-2 inhibitor for making a drug for treating cardiovascular pathologies resulting from ischemic situations.

2- The use of claim 1, wherein the caspase 2 inhibitor is a derivative of formula (I)
R—CO-A1-AspSubst-A-AspSubst-R1-R2 (I)
wherein
R is selected in the group comprising
a quinolin-2-yl group of formula II 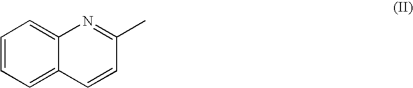
or,
substituted phenyl group of formula III 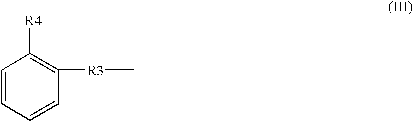
with R3 being —NH—CO— or —NH—CO—CH₂, and R4 being an alkyl group, preferably a branched alkyl group such as the tert-butyl group
A1 is Val, Leu, or is absent
AspSubst, is an aspartic acid residue of formula IV 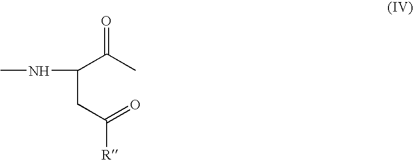
wherein R″ is
is O-alk, alk being a C1-C5 alkyl, or represents
“Linker-D”, with
“Linker” being —O— with one or several amino acids grafted thereon such as Gly or Gly-Phe-Leu-Gly-, or NH or NHCO, or CO—O—, or a malonyl group, and
“D” being
either a HPMA polymer (N-(2-hydroxypropyl)metheacrylamide polymer), or
Y, which represents a group of formula V 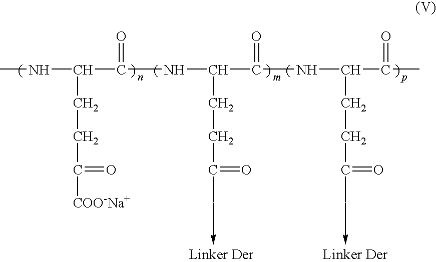
with n≧1; m≧1; p=0 or ≧1;
wherein “Der” means a derivative of formula I,
or R″ represents Z which is —(O)ₙ—PEG (polyethylene glycol=PEG100-100000; n=0-1)
or
—(O)ₙ—(CO)ₘ—C(CH₃)H—NH—CO—CH₂O-PEG-X
with X═OH or OCH₂CO₂H and PEG (polyethylene glycol=PEG100-100000) and n=0-1 and m=0-1
or
—(O)ₙ—(CO)ₘ—CH₂—NW—CO—CH₂—O-PEG-X (polyethylene glycol=PEG100-100000); n=0-1; m=0-1; W═H or CH₃; with X═OH or OCH₂CO₂H)
or
—(O)ₙ—(CO)ₘ—CH₂—NW-PEG-X (polyethylene glycol=PEG100-100000); n=0-1; m=0-1; W═H or alkyl; with X═OH or OCH₂CO₂H)
or
—(O)ₙ—(CO)ₘ—CH₂—O-PEG-X (polyethylene glycol=PEG100-100000); n=0 or 1; m=0-1; X═OH or OCH₂CO₂H)
or
—(O)ₙ—(CO)ₘ—CH₂—O—CH₂—CO—NW-PEG-X (polyethylene glycol=PEG100-100000); n=0-1; m=0-1; X═OH or OCH₂CO₂H; W═H or alkyl)
or
Z1-Der wherein Z1 is —(O)ₙCO—C(CH₃)H—NH—CO—CH₂O-PEG-CH₂—CO—NH—C(CH₃)H—CO—(O)ₙ—
with PEG=PEG 100-100000; n=0-1
or
—(O)ₙ—(CO)ₘ—C(CH₃)H—NH—CO—CH₂O-PEG-OCH₂—CO—NH—C(CH₃)H—(CO)ₘ—(O)ₙ—
with X═OH or OCH₂CO₂H and PEG (polyethylene glycol=PEG100-100000) and n=0-1 and m=0-1
or
—(O)ₙ—(CO)ₘ—CH₂—NW—CO—CH₂—O-PEG-O—CH₂—CO—NW—CH₂—(CO)ₘ—(O)ₙ—
with polyethylene glycol=PEG100 à 100000; n=0-1; m=0-1; W═H or CH₃; with X═OH or OCH₂CO₂H)
or
—(O)ₙ—(CO)ₘ—CH₂—NW-PEG-NW—CH₂—(CO)ₘ—(O)ₙ—
with polyethylene glycol=PEG100-100000; n=0-1; m=0-1; W═H or alkyl; with X═OH or OCH₂CO₂H
or
—(O)ₙ—(CO)ₘ—CH₂—O-PEG-O—CH₂—(CO)ₘ—(O)ₙ— (polyethylene glycol=PEG100-100000); n=0-1; m=0-1; X═OH or OCH₂CO₂H)
or
—(O)ₙ—(CO)ₘ—CH₂—O—CH₂—CO—NW-PEG-NW—CO—CH₂—O—CH₂—(CO)ₘ—(O)ₙ—
(polyethylene glycol=PEG100-100000); n=0-1; m=0-1; X═OH ou OCH₂CO₂H; W═H or alkyl)
and “Der” is as above defined.
or R″ represents J of formula VI 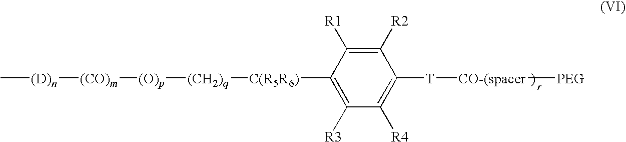
wherein
D=O or NH
n=0-1
m=0-1
p=0-1
q=0-1
i=0-1
r=0, 1-10
R1, R2, R3, R4=H or alkyl
R5, R6=H or alkyl
Spacer=one amino acid (for instance, alanine, proline, β-alanine, NH(CH₂CH₂O)₂, NH(CH₂CH₂O)CH₂CH₂NH
T=O or NH
PEG=PEG100-100000
A is
either A2-A3, with A2 being Val or Glu and A3 being Ala, Ser, Tic (1,2,3,4-tetrahydroisoquinoline-3-carbonyl) and Aic (2-amino-2,3-dihydro-1H-indene-2-carbony),
or
A2-A3 being 3-amino-4-oxo-1,2,3,4,6,7-hexahydroazepino[3,2,1-hi] indole-6-carbonyl,
R1 is selected in the group comprising —CH₂O—,
R2 is a phenyl group substituted by one or several groups, identical or different, selected amongst the halogen atoms and/or alkyl, alkoxy, carboxyl, 1-oxoalkyl groups
and the pharmaceutically acceptable salts thereof, and the stereoisomeric forms (enantiomers and diastereoisomers) or the racemic mixtures.

3- The use of claim 2, wherein A1 and A2 are a valine residue.

4- The use of claim 2, wherein A1 is a valine residue and A2 is a glutamic acid residue.

5- The use of anyone of claims 2 to 4, wherein AspSubst in formula (I) is an aspartyl residue with the carboxyl group substituted by a OCH₃ group.

6- The use of anyone of claims 1 to 5, wherein R2 a phenyl group substituted by 2 to 5 fluor.

7- The use according to anyone of claims 1 to 6, wherein the derivative is selected in the group comprising
D1: (3S,6S,9S,12S)-methyl 15-(2-(2,6-difluorophenoxy)acetyl)-3,9-diisopropyl-6-(2-methoxy-2-oxoethyl)-12-methyl-1,4,7,10,13-pentaoxo-1-(quinolin-2-yl)-2,5,8,11,14-pentaazaheptadecan-17-oate of formula VII 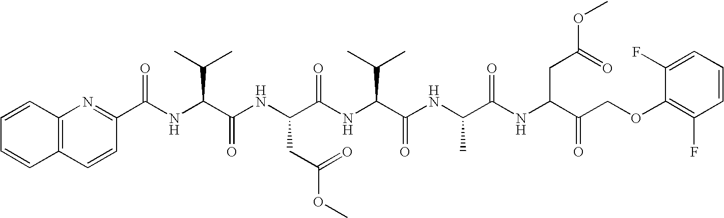
(VII)
D2: methyl 5-(2,6-difluorophenoxy)-3-((S)-2-((S)-2-((S)-4-methoxy-2-4S)-3-methyl-2-(quinoline-2-carboxamido)butanamido)-4-oxobutanamido)-3-methylbutanoyl)-1,2,3,4-tetrahydroisoquinoline-3-carboxamido)-4-oxopentanoate of formula VIII 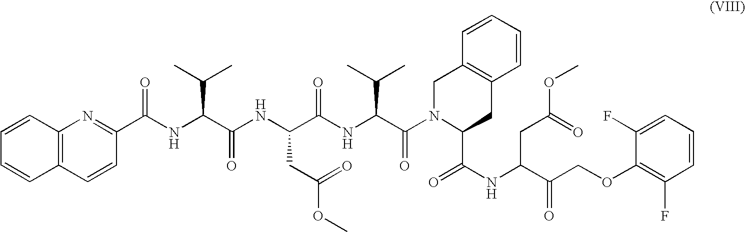
D3: methyl 5-(2,6-difluorophenoxy)-3-(2-((S)-2-((S)-4-methoxy-2-((S)-3-methyl-2-(quinoline-2-carboxamido)butanamido)-4-oxobutanamido)-3-methylbutanamido)-2,3-dihydro-1H-indene-2-carboxamido)-4-oxopentanoate of formula IX 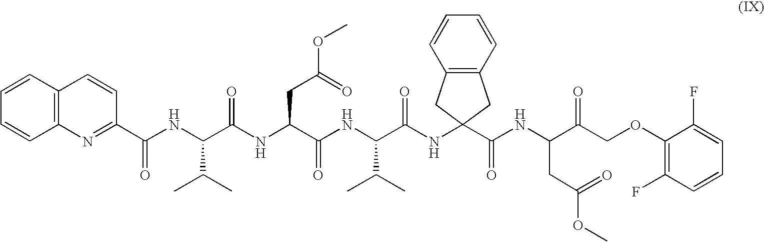
D4: (4S)-5-((2S)-1-(5-(2,6-difluorophenoxy)-1-methoxy-1,4-dioxopentan-3-ylamino)-1-oxopropan-2-ylamino)-4-((S)-4-methoxy-2-((S)-3-methyl-2-(quinoline-2-carboxamido)butanamido)-4-oxobutanamido)-5-oxopentanoic acid of formula X 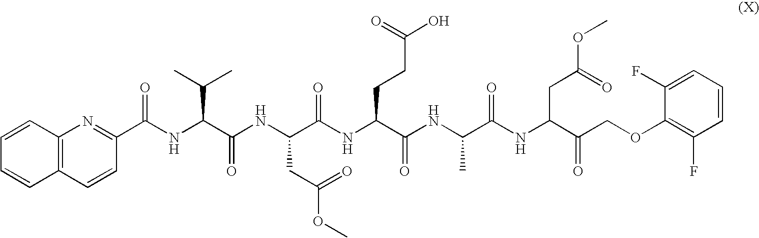
D5: (4S)-5-((2S)-1-(5-(2,6-difluorophenoxy)-1-methoxy-1,4-dioxopentan-3-ylamino)-3-hydroxy-1-oxopropan-2-ylamino)-4-((S)-4-methoxy-2-((S)-3-methyl-2-(quinoline-2-carboxamido)butanamido)-4-oxobutanamido)-5-oxopentanoic acid of formula XI 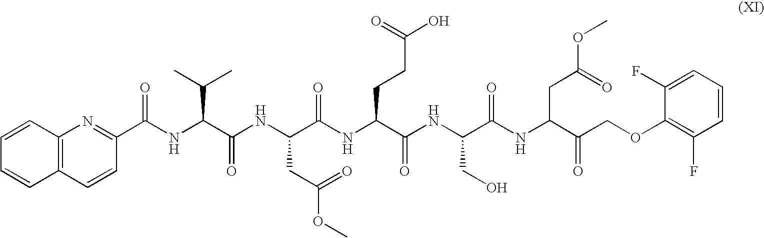
D6: (3 S,6 S,9 S,12 S)-methyl 3,9-diisopropyl-6-(2-methoxy-2-oxo ethyl)-12-methyl-1,4,7,10,13-pentaoxo-1-(quinolin-2-yl)-15-(2-(2,3,5,6-tetrafluorophenoxy)acetyl)-2,5,8,11,14-pentaazaheptadecan-17-oate of formula XII 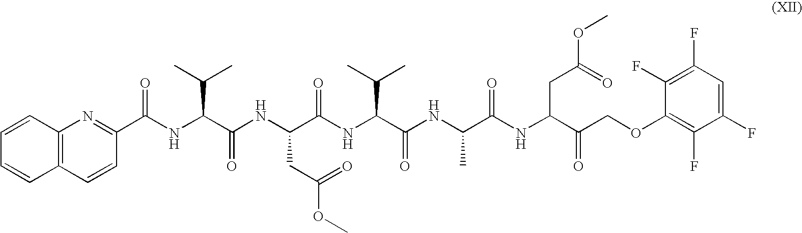
D7: methyl 5-(2,6-difluorophenoxy)-3-((3S,6S)-3-((S)-4-methoxy-2-((S)-3-methyl-2-(quinoline-2-carboxamido)butanamido)-4-oxobutanamido)-4-oxo-1,2,3,4,6,7-hexahydroazepino[3,2,1-hi]indole-6-carboxamido)-4-oxopentanoate of formula XIII 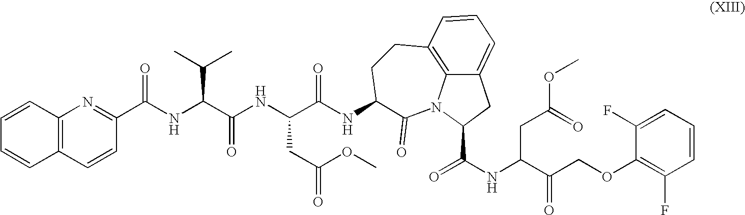
D8: (4S,7S,10S,13S)-methyl 1-(2-tert-butylphenylamino)-16-(2-(2,6-difluorophenoxy)acetyl)-4,10-diisopropyl-7-(2-methoxy-2-oxoethyl)-13-methyl-1,2,5,8,11,14-hexaoxo-3,6,9,12,15-pentaazaoctadecan-18-oate of formula XIV 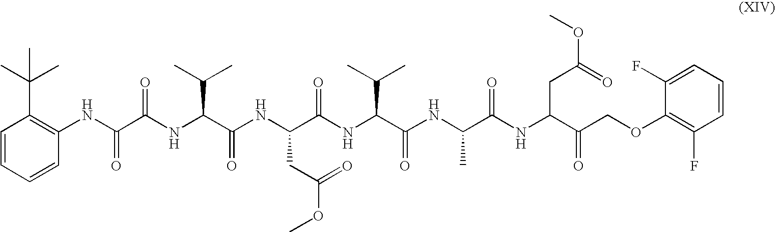
D9: (4S,7S,10 S,13S)-methyl 1-(2-tert-butylphenylamino)-4,10-diisopropyl-7-(2-methoxy-2-oxo ethyl)-13-methyl-1,2,5,8,11,14-hexaoxo-16-(2-(2,3,5,6-tetrafluorophenoxy)acetyl)-3,6,9,12,15-pentaazaoctadecan-18-oate of formula XV 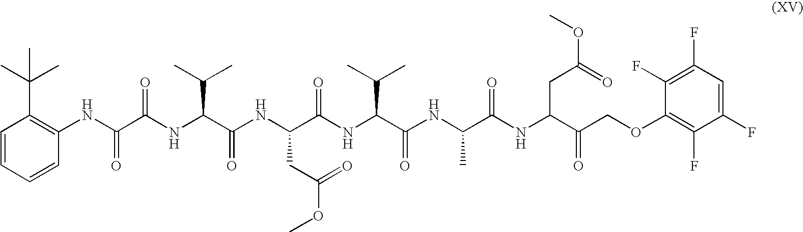
D10: (4 S,7 S,10 S)-methyl 1-(2-tert-butylphenylamino)-13-(2-(2,6-difluorophenoxy)acetyl)-7-isopropyl-4-(2-methoxy-2-oxo ethyl)-10-methyl-1,2,5,8,11-pentaoxo-3,6,9,12-tetraazapentadecan-15-oate of formula XVI 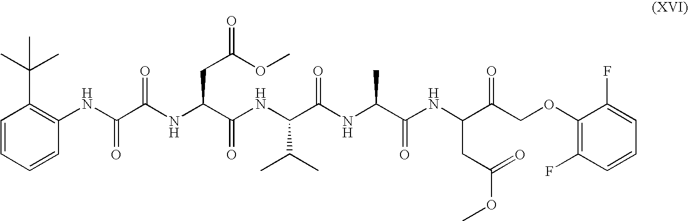
D11: (4 S,7 S,10S)-methyl 1-(2-tert-butylphenylamino)-7-isopropyl-4-(2-methoxy-2-oxo ethyl)-10-methyl-1,2,5,8,11-pentaoxo-13-(2-(2,3,5,6-tetrafluorophenoxy)acetyl)-3,6,9,12-tetraazapentadecan-15-oate of formula XVII 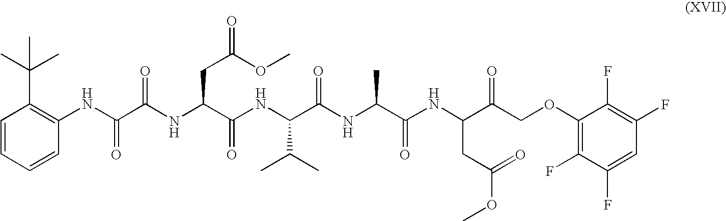
D12: (6S,9S,12S,15S)-methyl 19-(2-tert-butylphenylamino)-3-(2-(2,6-difluorophenoxy)acetyl)-9,15-diisopropyl-12-(2-methoxy-2-oxo ethyl)-6-methyl-5,8,11,14,17,19-hexaoxo-4,7,10,13,16-pentaazanonadecan-1-oate of formula XVIII 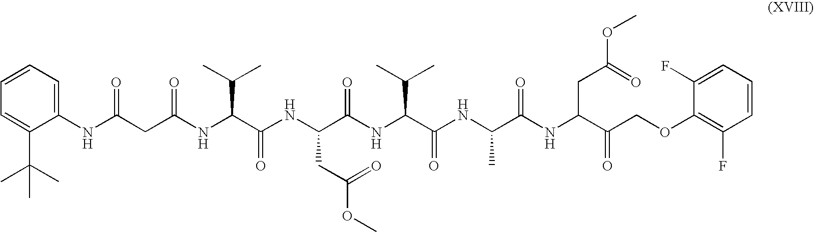
D13: (6 S,9 S,12 S,15S)-methyl 19-(2-tert-butylphenylamino)-9,15-diisopropyl-12-(2-methoxy-2-oxo ethyl)-6-methyl-5,8,11,14,17,19-hexaoxo-3-(2-(2,3,5,6-tetrafluorophenoxy)acetyl)-4,7,10,13,16-pentaazanonadecan-1-oate of formula XIX 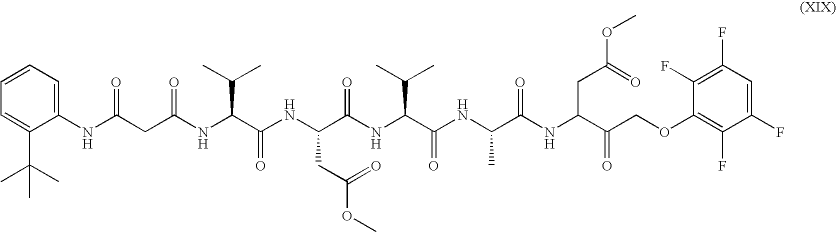
D14: (4S)-5-((2S)-1-(5-(2,6-difluorophenoxy)-1-methoxy-1,4-dioxopentan-3-ylamino)-1-oxopropan-2-ylamino)-4-((S)-4-methoxy-2-((S)-4-methyl-2-(quinoline-2-carboxamido)pentanamido)-4-oxobutanamido)-5-oxopentanoic acid of formula XX 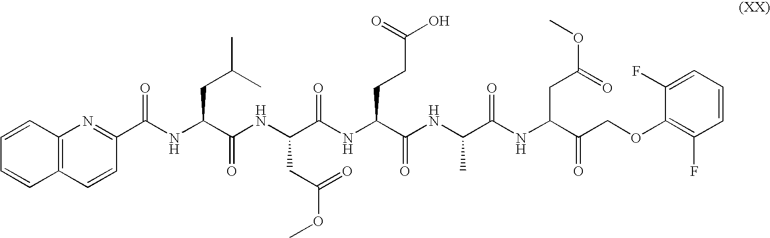
D15: (4S)-5-((2S)-1-(1-methoxy-1,4-dioxo-5-(2,3,5,6-tetrafluorophenoxy)pentan-3-ylamino)-1-oxopropan-2-ylamino)-4-((S)-4-methoxy-2-((S)-4-methyl-2-(quinoline-2-carboxamido)pentanamido)-4-oxobutanamido)-5-oxopentanoic acid of formula XXI 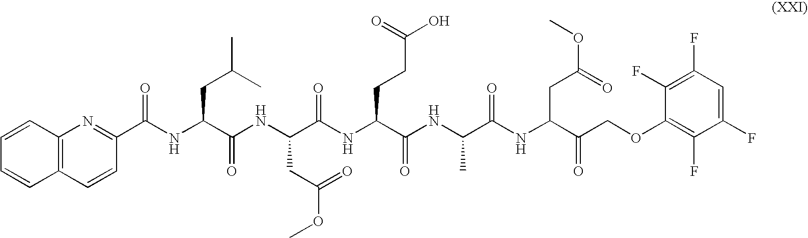
D16: N-(2-hydroxypropyl)methacrylamide copolymer-TRP601 (with A=D1), said derivative 18 having formula XXII 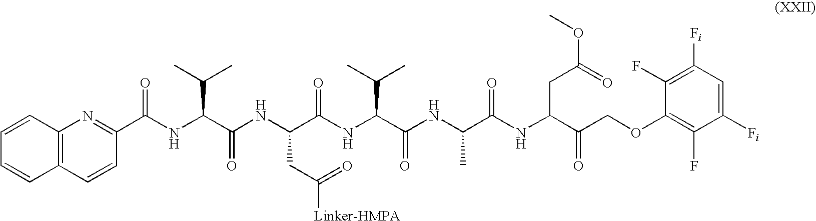
with
Linker=
one or several amino acids (Gly or Gly-Phe-Leu-Gly for example) grafted on the carboxylic function of the P4 Asp side-chain via an amide or ester function
a malonate derivative
i=0-1, with Fᵢ=H when i=0 and Fᵢ=F when Fᵢ=1
HPMA=N-(2-hydroxypropyl)methacrylamide polymer (n≧1; m≧1) 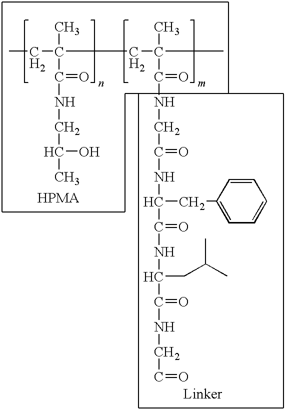
D17: Asp-Linker-Y polyglutamate-TRP601 (with A=D1), said derivative 19 having of formula XXIII 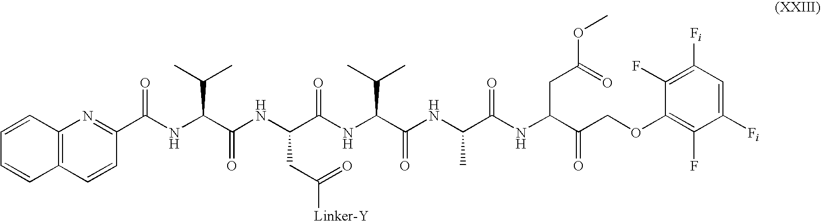
with
i=0-1, with Fᵢ=H when i=0 and Fᵢ=F when Fᵢ=1
Linker=one or several amino acids grafted on the COOH group of the P4 Asp side-chain.
Y= 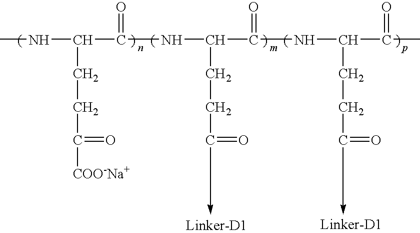
with n≧1; m≧1; p=0 or ≧1; i=0-1 and D1 is as above defined
D18: N^{α}-Quinoline-2-carbonyl-(S)-Val-(S)-Asp(Z)-(S)-Val-(S)-Ala-(R,S)-Asp(OMe)-CH₂O—C₆H₃-2,6-F₂ or N^{α}-Quinoline-2-carbonyl-(S)-Val-(S)-Asp(Z)-(S)-Val-(S)-Ala-(R,S)-Asp(OMe)-CH₂O—C₆H-2,3,5,6-F₄ of formula XXIV 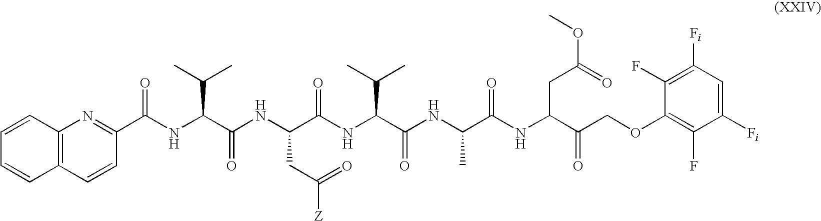
Wherein:
i=0-1, with Fᵢ=H for i=0 and Fᵢ=F for i=1
and
Z=
—(O)ₙ—PEG (polyethylene glycol=PEG100-100000; n=0-1)
or
—(O)ₙ—(CO)ₘ—C(CH₃)H—NH—CO—CH₂O-PEG-X
with X═OH or OCH₂CO₂H and PEG (polyethylene glycol=PEG100-100000) and n=0-1 and m=0-1
or
—(O)ₙ—(CO)ₘ—CH₂—NW—CO—CH₂—O-PEG-X (polyethylene glycol=PEG100 à 100000);
n=0-1; m=0-1; W═H or CH₃; with X═OH or OCH₂CO₂H
or
—(O)ₙ—(CO)ₘ—CH₂—NW-PEG-X (polyethylene glycol=PEG100-100000); n=0-1; m=0-1;
W═H or alkyl; with X═OH or OCH₂CO₂H
or
—(O)ₙ—(CO)ₘ—CH₂—O-PEG-X (polyethylene glycol=PEG100-100000); n=0 or 1; m=0-1;
X═OH or OCH₂CO₂H
or
—(O)ₙ—(CO)ₘ—CH₂—O—CH₂—CO—NW-PEG-X (polyethylene glycol=PEG100-100000);
n=0-1; m=0-1; X═OH or OCH₂CO₂H; W═H or alkyl
D19: TRP601-PEG-TRP601-(with A=D1), said derivative 21 having formula XXV 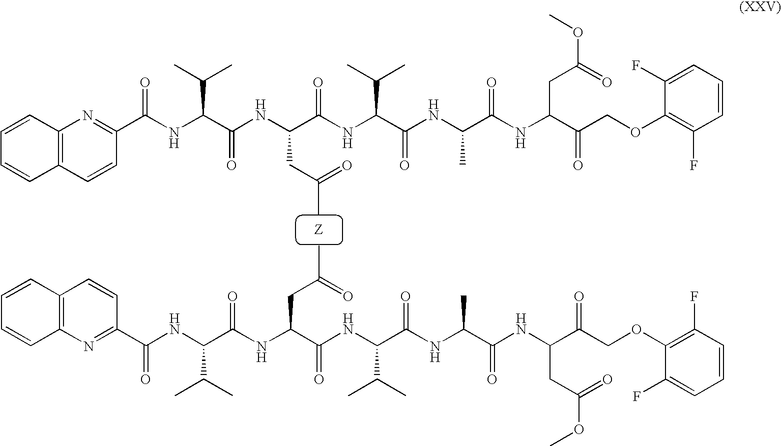
wherein Z=
—(O)ₙCO—C(CH₃)H—NH—CO—CH₂O-PEG-CH₂—CO—NH—C(CH₃)H—CO—(O)ₙ—
with PEG=PEG 100-100000; n=0-1
or
—(O)ₙ—(CO)ₘ—C(CH₃)H—NH—CO—CH₂O-PEG-OCH₂—CO—NH—C(CH₃)H—(CO)ₘ—(O)ₙ—
with X═OH or OCH₂CO₂H and PEG (polyethylene glycol=PEG100-100000); n=0-1 and m=0-1
or
—(O)ₙ—(CO)ₘ—CH₂—NW—CO—CH₂—O-PEG-O—CH₂—CO—NW—CH₂—(CO)ₘ—(O)ₙ—
with polyethylene glycol=PEG100-100000; n=0-1; m=0-1; W═H or CH₃; with X═OH or OCH₂CO₂H)
or
—(O)ₙ—(CO)ₘ—CH₂—NW-PEG-NW—CH₂—(CO)ₘ—(O)ₙ—
with polyethylene glycol=PEG100-100000; n=0-1; m=0-1; W═H or alkyl; with X═OH or OCH₂CO₂H
or
—(O)ₙ—(CO)ₘ—CH₂—O-PEG-O—CH₂—(CO)ₘ—(O)ₙ— (polyethylene glycol=PEG100-100000);
n=0-1; m=0-1; X═OH or OCH₂CO₂H
or
—(O)ₙ—(CO)ₘ—CH₂—O—CH₂—CO—NW-PEG-NW—CO—CH₂—O—CH₂—(CO)ₘ—(O)ₙ—
(polyethylene glycol=PEG100-100000); n=0-1; m=0-1; X═OH or OCH₂CO₂H; W═H, CH₃ or alkyl
D20: N^{α}-Quinoline-2-carbonyl-(S)-Val-(S)-Asp(J)-(S)-Val-(S)-Ala-(R,S)-Asp(OMe)-CH₂O—C₆H₃-2,6-F₂ or N^{α}-Quinoline-2-carbonyl-(S)-Val-(S)-Asp(J)-(S)-Val-(S)-Ala-(R,S)-Asp(OMe)-CH₂O—C₆H-2,3,5,6-F₄ of formula XXVI 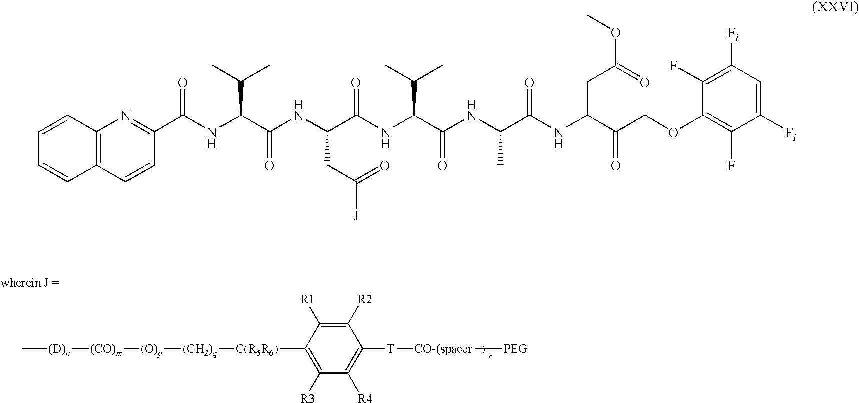
D=O or NH
n=0-1
m=0-1
p=0-1
i=0-1, with Fᵢ=H for i=0 and Fᵢ=F for i=1
r=0, 1-10
R1, R2, R3, R4=H or alkyl
R5, R6=H or alkyl
Spacer=one amino acid (for example, alanine, proline, β-alanine, NH(CH₂CH₂O)₂, NH(CH₂CH₂O)CH₂CH₂NH
T=O or NH
PEG=PEG100-100000

8- New peptides having formula I of claim 1, corresponding to derivatives D6 to D20 of claim 7.

9- The peptides of claims 8 for use as drugs.

10- Pharmaceutical compositions comprising therapeutically effective amount of at least one compound of formula I, except D1 to D5 of claim 8, in association with a pharmaceutically acceptable vehicle.

11- The use according to anyone of claims 1 to 7, and the pharmaceutical of claim 10, wherein the derivatives are under a form suitable for an administration by intravenous route or intramuscular or subcutaneous.

12- The use according to anyone of claim 1 to 7 or 11, and the pharmaceutical compositions of claim 10 for the treatment of lesions and ischemic cardiopathies.

13- The use of claim 12 and the pharmaceutical compositions of claim 10, for the treatment of myocardial infarction, coronary cardiopathies and cardiac deficiencies.

14- The use of claim 12 and the pharmaceutical compositions of claim 10, for the treatment of processus having a strong inflammatory component or oxidative stress component, at the brain level in adults and in neonates (global or focal cerebral ischemia, asphyxia, hypoxia-ischemia, traumatic brain injury), or in the eye, internal ear, kidney.

15- The use of claim 12 and the pharmaceutical compositions of claim 10, for the protection of grafts during heart, liver, skin and kidney transplants.
